(19) United States
(12) Patent Application Publication
(10) Pub. No.: US 2021/0261621 A1
(43) Pub. Date: Aug. 26, 2021

(54) Preparation of Dilipid Polymixins and Use Thereof as Antimicrobial Adjuvants

(71) Applicant: UNIVERSITY OF MANITOBA, Winnipeg (CA)

(72) Inventors: Ronald Domalaon, Winnipeg (CA); Frank Schweizer, Winnipeg (CA)

(21) Appl. No.: 16/972,700

(22) PCT Filed: Jun. 5, 2019
(86) PCT No.: PCT/CA2019/050780

§ 371 Date: Dec. 7, 2020

Related U.S. Application Data
(60) Provisional application No. 62/683,847, filed on Jun. 12, 2018.

(51) Int. Cl. C07K 7/62; A61P 31/04

(52) U.S. Cl. CPC C07K 7/62; A61K 38/00; A61P 31/04

(57) ABSTRACT

A secondary fatty acyl component of varying length was added onto the polymyxin structure at the amine side-chain of L-diaminobuytric acid at position 1, resulting to the development of dilipid polymyxins. The incorporation of an additional lipid was found to confer to polymyxin activity against Gram-positive bacteria, to which polymyxins are inherently inactive against The dilipid polymyxins showed selective antibacterial activity against Pseudomonas aeruginosa. Moreover, dilipid polymyxin 1 that consists of four carbon-long aliphatic lipids displayed the ability to enhance the antibacterial potency of other antibiotics in combination against P. aeruginosa, thereby resembling the adjuvant activity of the well-known outer membrane permeabilizer polymyxin B nonapeptide (PMBN).

### PRIOR APPLICATION INFORMATION

The instant application claims the benefit of U.S. Provisional Patent Application 62/683,847, filed Jun. 12, 2018 and entitled “PREPARATION OF DILIPID POLYMYXINS AND USE THEREOF AS ANTIMICROBIAL ADJUVANTS”, the entire contents of which are incorporated herein by reference.

### BACKGROUND OF THE INVENTION

Polymyxins are a class of antibacterials used as drugs of last resort to treat multidrug-resistant (MDR) Gram-negative bacterial infections that are non-responsive to conventional antibiotic treatments [1]. Concerns with polymyxin's nephrotoxicity and neurotoxicity as well as the availability of less toxic alternatives hampered their widespread clinical usage in the past. However, a rejuvenated interest in polymyxins have been observed recently due to the alarming increase of MDR pathogens that are impervious to most antibiotics, but also due to improved understanding of polymyxin's pharmacokinetic/pharmacodynamic properties and how they relate to alleviating toxicity [1,2]. Polymyxin B and E, also known as colistin, (FIG. 1A) are currently used in the clinic as monotherapy or as part of combination therapy with other antibiotics when standard treatment options fail [3].

The structure of polymyxins consist of a cyclic heptapeptide core attached to a linear tripeptide with an acylated N-terminus to a fatty acid (FIG. 1A). Segregated hydrophobic and hydrophilic domains bestow polymyxins an amphiphilic nature. Polymyxin's hydrophilicity is due to the polar amine side-chains of L-2,4,-diaminobutyric acid (Dab) and hydroxyl side-chains of L-threonine (Thr). These polar side-chains, but also the peptide/amide backbone, are responsible for polymyxins' lipopolysaccharide (LPS) binding that consequently result to displacement of divalent cation bridges and outer membrane destabilization [4]. The majority of polymyxins' hydrophobic character is from the fatty acid acylated to its N-terminus. This lipid component is believed to be crucial in polymyxins' insertion into the destabilized outer membrane and its transit to periplasmic space [5], but also is responsible for disrupting the inner membrane stability [6,7]. Hydrophobicity is also imparted by L-leucine (Leu) and either D-phenylalanine (phe) in polymyxin B or D-leucine (leu) in colistin. These hydrophobic amino acids are believed to play a crucial role in LPS binding [4]. Overall, the amphiphilic nature of polymyxins result to membrane disruption that leads to intracellular component leakage and bacterial cell death. Notably, polymyxins exhibit poor activity against Gram-positive bacteria as they do not bind favorably to lipoteichoic acid studded in the cytoplasmic membrane [8].

Structure-activity relationship (SAR) studies have generated a wealth of knowledge on the parameters which are critical/non-critical for polymyxins' antibacterial activity. For instance, alanine scanning of polymyxin B revealed several amino acid side-chains that are not crucial for activity [9]. It also has been elucidated that aliphatic hydrocarbon lipids of seven to nine carbons-long are optimal for antibacterial activity [4]. However, several aliphatic hydrocarbon non-classical isosteres such as adamantyl and aromatic functional groups may yield derivatives with similar antibacterial activity to polymyxins but with less nephrotoxicity [4,10]. Removal of the lipid and Dab at position 1 (Dab1) yields polymyxin B nonapeptide (PMBN) (FIG. 1B), which is known to permeabilize the outer membrane yet lacks the ability to kill bacteria [11]. In fact, PMBN is known to enhance the cellular entry and therefore antibacterial activity of antibiotics that have limited outer membrane penetration [12]. The absence of antibacterial activity in PMBN is due to the lack of lipid component crucial for interaction of polymyxins with lipid bilayers of the outer- and inner membranes of Gram-negative bacteria. Several SAR studies confirmed that hydrophobicity at certain structural points of PMBN is crucial for its outer membrane sensitization and LPS binding properties [13,14]. A derivative of PMBN called SPR741 is currently being evaluated in clinical studies as an adjuvant to enhance the efficacy of antibiotics in combination against Gram-negative pathogens [15,16]. For instance, SPR741 potentiated an extensive panel of antibiotics against Enterobacteriacaea and Acinetobacter baumannii, but not against Pseudomonas aeruginosa [17]. Two recently completed phase-1 clinical studies showed SPR741 to be well-tolerated in healthy volunteers up to a single dose of 800 mg or multiple dose up to 600 mg every 8 hours for 14 consecutive days.

### SUMMARY OF THE INVENTION

According to an aspect of the invention, there is provided a compound comprising a chemical structure as set forth in formula (I):

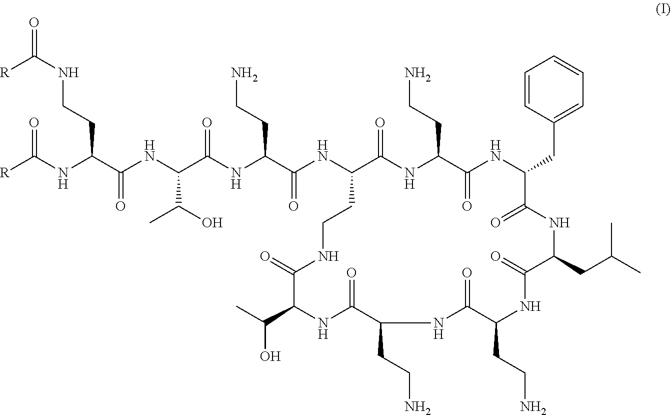

wherein: each R is independently a substituted or unsubstituted aliphatic lipid of C1-8.

According to another aspect of the invention, there is provided use of a compound comprising a chemical structure as set forth in formula (I)

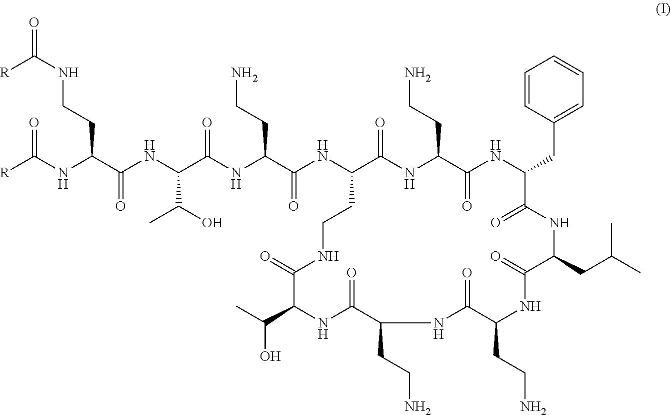

wherein: each R is independently a substituted or unsubstituted aliphatic lipid of C1-8, as an anti-microbial adjuvant.

According to a further aspect of the invention, there is provided a method of treating a microbial infection comprising: administering to an individual in need of such treatment an effective amount of an antimicrobial agent and an effective amount of a compound comprising a chemical structure as set forth in Formula (I):

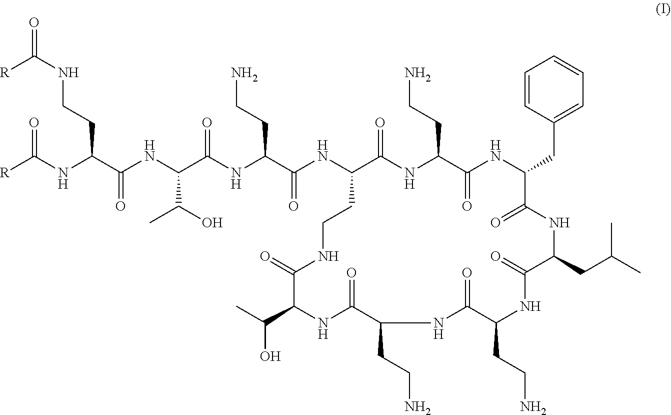

wherein: each R is independently a substituted or unsubstituted aliphatic lipid of C1-8.

According to another aspect of the invention, there is provided a method of increasing activity of an anti-microbial agent against a target micro-organism comprising co-administering to the micro-organism a compound comprising a chemical structure as set forth in Formula (I)

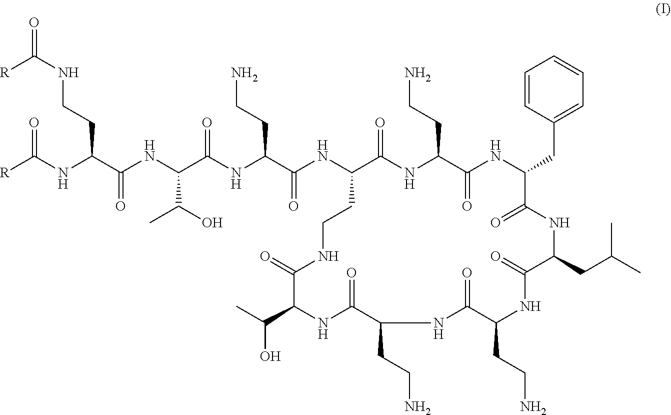

wherein: each R is independently a substituted or unsubstituted aliphatic lipid of C1-8, as an anti-microbial adjuvant, and an effective amount of the anti-microbial agent.

### BRIEF DESCRIPTION OF THE DRAWINGS

FIG. 1. Polymyxin structures: (A) polymyxin B & colistin; (B) polymyxin B nonapeptide (PMBN).

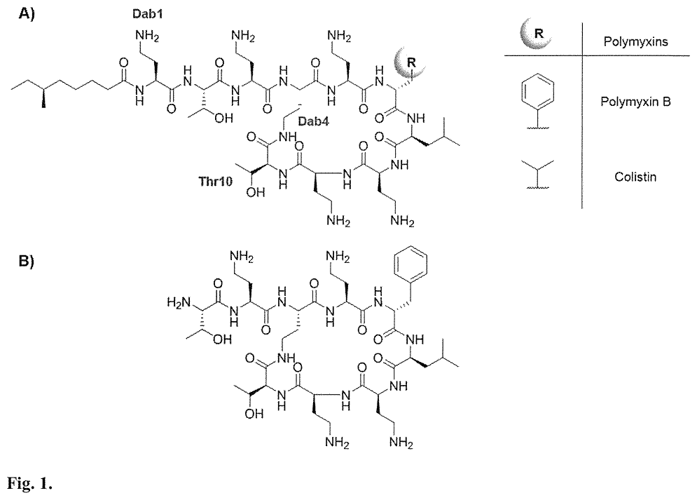

FIG. 2. Synthesized dilipid polymyxins.

FIG. 3. Concentration-dependent hemolysis of red blood cells induced by dilipid polymyxins, colistin and polymyxin B nonapeptide. Yellow circle=dilipid polymyxin 1; yellow triangle=dilipid polymyxin 2; red circle=dilipid polymyxin 3; red triangle=dilipid polymyxin 4; green circle=dilipid polymyxin 5; green triangle=colistin; blue circle=polymyxin B nonapeptide (PMBN). Experiment performed in at least triplicates.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Continuous development of new antibacterial agents is necessary to counter the problem of antimicrobial resistance. Polymyxins are considered as drugs of last resort to combat multidrug-resistant Gram-negative pathogens. Structural optimization of polymyxins requires an in-depth understanding of its structure and how it relates to its antibacterial activity. Herein, the effect of hydrophobicity was explored by adding a secondary fatty acyl component of varying length onto the polymyxin structure at the amine side-chain of L-diaminobuytric acid at position 1, resulting to the development of dilipid polymyxins. The incorporation of an additional lipid was found to confer to polymyxin activity against Gram-positive bacteria, to which polymyxins are inherently inactive against. The dilipid polymyxins showed selective antibacterial activity against Pseudomonas aeruginosa. Moreover, dilipid polymyxin 1 that consists of four carbon-long aliphatic lipids displayed the ability to enhance the antibacterial potency of other antibiotics in combination against P. aeruginosa, thereby resembling the adjuvant activity of the well-known outer membrane permeabilizer polymyxin B nonapeptide (PMBN). Interestingly, our data revealed that dilipid polymyxin 1 and PMBN are substrates for the MexAB-OprM efflux system, and therefore are affected by efflux. In contrast, dilipid polymyxin analogs that consist of longer lipids and colistin were not affected by efflux, suggesting that the lipid component of polymyxin plays an important role in resisting active efflux. Our work described herein provides an understanding to the polymyxin structure that may be used to usher the development of enhanced polymyxin analogs.

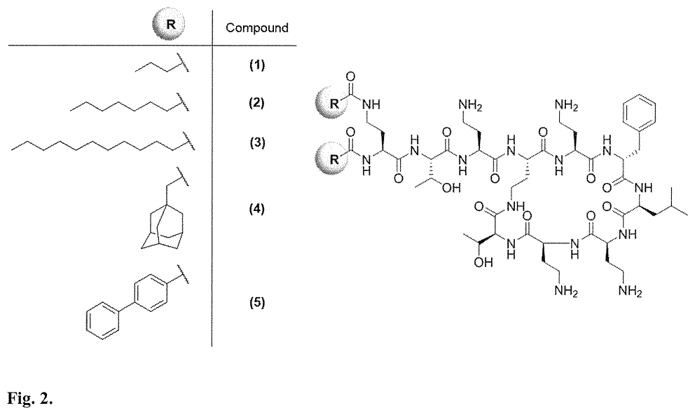

It is evident that hydrophobicity plays a critical role in the antibacterial activity of polymyxins [4]. A recent study revealed that remodelling of outer membrane through pagL-induced lipid A deacylation resulted to decreased membrane interaction and penetration of polymyxins [18]. The removal of an acyl group from the typically hexa-acylated lipid A occurred in the presence of sub-inhibitory polymyxin concentrations in both polymyxin-susceptible and -resistant organisms, resulting to a more efficient lipid packing that prevent polymyxins from inserting into the bilayer and traversing onto the periplasm [18]. We hypothesize that the addition of hydrophobic functional groups into the polymyxin structure may enhance its ability to insert into membranes and therefore enhance its activity against Gram-negative but also Gram-positive bacteria. An effort to elucidate the effect of hydrophobicity on polymyxin's antipseudomonal activity by replacing the primary amine side-chain of Dab to tertiary N,N-dimethylamine was recently reported [19]. In this study, we explore the effect of adding hydrophobicity to the polymyxin structure by acylating the amine side-chain of Dab1 with different fatty acids. These dilipid polymyxins (FIG. 2) were then evaluated for their antibacterial activity against wild-type and multidrug-resistant clinical isolates of Gram-positive and Gram-negative bacteria. Moreover, their ability to potentiate the efficacy of other antibiotics in combination, as observed with PMBN, was also assessed. Furthermore, we explored whether the lipid component affect the ability of polymyxin to resist active efflux. To address the concern that an enhanced hydrophobicity may result to non-specific lysis of membranes including eukaryotic membranes, we measured the dilipid polymyxin's lytic activity against red blood cells. Our study provides valuable information for optimization of the polymyxin class of antibiotics as stand-alone antibiotics but also as antimicrobial adjuvant to potentiate other classes of antibiotics.

Our efforts herein have elucidated several important key points useful for the rational optimization of the polymyxin structure. The addition of an extra lipid component conferred polymyxin activity against Gram-positive bacteria and selectivity against Gram-negative bacteria. Similar to PMBN, the dilipid polymyxins can also enhance the activity of other antibiotics in combination. The lipid component appeared to modulate the ability of polymyxins to resist efflux, where derivatives acylated with shorter fatty acids and PMBN are greatly affected by the MexAB-OprM efflux system. However, lipid component of longer fatty acids may promote unwanted non-specific lysis and therefore cytotoxicity. Aliphatic lipids of eight carbons or less, caged or aromatic hydrocarbons are preferred. Overall, these data provide useful insights that may guide the optimization of the polymyxin structure.

According to an aspect of the invention, there is provided a compound comprising a chemical structure as set forth in formula (I):

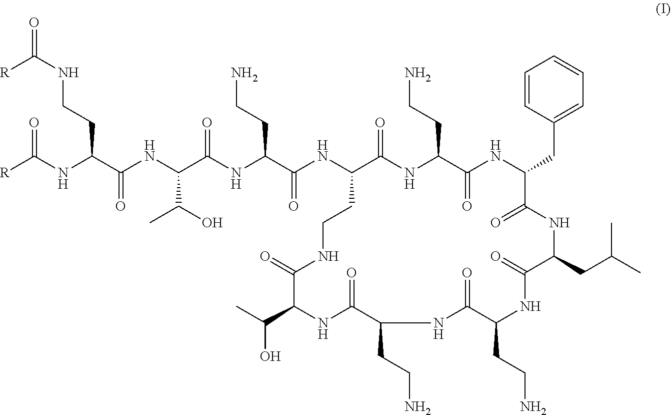

wherein: each R is independently a substituted or unsubstituted aliphatic lipid of C1-8.

In some embodiments of the invention, the substituted or unsubstituted aliphatic lipid is C1-C7. In other embodiments of the invention, the aliphatic lipid may be C1-C6, C1-C5, C1-C4, C1-C3, C1-C2, C2-C8, C2-C7, C2-C6, C2-C5, C2-C4, C2-C3, C3-C8, C3-C7, C3-C6, C3-C5, C3-C4, C4-C8, C4-C7, C4-C6, C4-C5, C5-C8, C5-C7, C5-C6, C6-C8, C6-C7 or C7-C8.

In some embodiments of the invention, the aliphatic lipids are substituted by replacing the alkyl chains with isosteric caged or aromatic moieties. As discussed herein, these substitutions lower hemolysis.

In some embodiments of the invention, the compound is for use as an anti-bacterial adjuvant.

As discussed herein, the compounds of the invention can be co-administered with a clinically-used antibiotic for inhibiting microbial growth.

As used herein, “administered” may refer to administration of the compound and a known antimicrobial agent to an individual in need of such treatment, as discussed below. Alternatively, the compound may also be “administered” to a surface at risk of microbial infection as an “anti-infective”. As will be appreciated by one of skill in the art, the adjuvant compounds of the invention may be combined with or added to any suitable “anti-infective” composition for enhancing or increasing the anti-microbial activity of the other compounds. Examples include but are by no means limited to cleaning products, anti-microbial coatings and the like.

A such, according to another aspect of the invention, there is provided use of a compound comprising a chemical structure as set forth in formula (I)

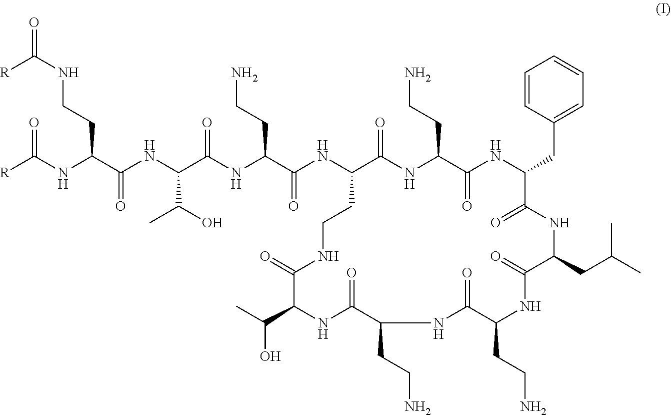

wherein: each R is independently a substituted or unsubstituted aliphatic lipid of C1-8, as an anti-microbial adjuvant.

For example, the compound may be used in combination with a known anti-microbial compound for increasing or enhancing or improving the activity of the known anti-microbial agent against the target organism. As will be appreciated by one of skill in the art, the target organism is the organism that is causing the microbial infection or that is suspected of causing the microbial infection.

It is of note that the known antimicrobial agent does not necessarily need to have activity against the target organism because, as discussed herein, the adjuvant compounds of the invention have been demonstrated to alter the susceptibility of certain organisms to specific antimicrobials.

As used herein, “co-administered” may refer to the adjuvant compound and the anti-microbial agent being administered simultaneously to an individual in need of such treatment, that is, to an individual who has been diagnosed with a microbial infection or an individual who is suspected of having a microbial infection. Alternatively, “co-administered” may mean that either the anti-microbial agent or the adjuvant compound of the invention is administered first and the other is administered soon thereafter. As will be appreciated by one of skill in the art, the administration must be “soon” enough that the antimicrobial agent can take advantage of the membrane permeabilization effected by the adjuvant compounds of the invention. As will be appreciated by one of skill in the art, this will depend on several factors, including but by no means limited to the target organism and the nature of and severity of the infection.

The adjuvant compounds of the invention may be co-administered with any suitable antimicrobial agent. Examples include but are by no means limited to β-lactams, carbapenems, tetracyclines, aminoglycosides, fluoroquinones, Fosfomycin, trimethoprim, chloramphenicol, novobiocin, vancomycin, clindamycin, linezolid, pleuromutilin and rifampicin. It is of note that other suitable anti-microbials will be readily apparent to one of skill in the art.

Alternatively, as discussed above, the compounds of the invention may be combined with or added to any suitable “anti-infective” composition for enhancing or increasing the anti-microbial activity of the other compounds. Examples include but are by no means limited to cleaning products, anti-microbial coatings and the like.

According to another aspect of the invention, there is provided a method of treating a microbial infection comprising: administering to an individual in need of such treatment an effective amount of an antimicrobial agent and an effective amount of a compound comprising a chemical structure as set forth in Formula (I):

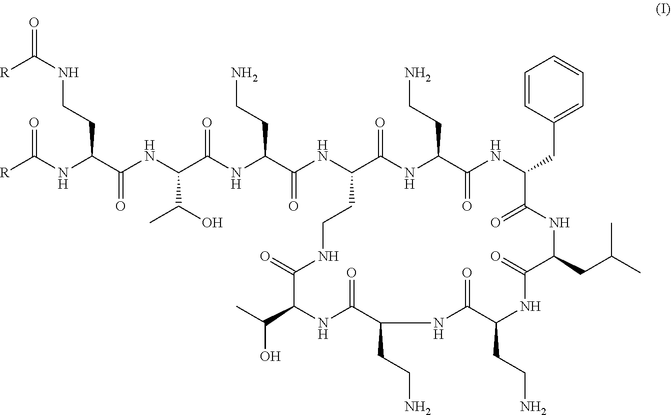

wherein: each R is independently a substituted or unsubstituted aliphatic lipid of C1-8.

As used herein, “a person in need of such treatment” is an individual who is known to have or who is suspected of having a microbial infection.

As used herein, an effective amount of the antimicrobial agent is an amount that is sufficient to have at least one of the following effects: reduction in colony forming units per ml of the micro-organism; reduction in total number of cells of the micro-organism within the host or individual; and reducing the severity of one or more symptoms associated with the microbial infection.

It is of note that such an effective amount of a specific anti-microbial agent can be easily determined through routine experimentation or may already be known. Furthermore, as discussed herein, it is important to note that such an effective amount may in fact be lower that what is currently considered to be necessary by virtue of the effect of the addition of the adjuvant compound of the invention, as discussed herein.

Accordingly, an effective amount of the adjuvant compound of the invention is an amount of the adjuvant compound that is sufficient to increase membrane permeability of the membrane of the target microorganism so that the anti-microbial agent can enter the microorganism.

In another aspect of the invention, there is provided a method of increasing activity of an anti-microbial agent against a target micro-organism comprising co-administering to the target micro-organism a compound comprising a chemical structure as set forth in Formula (I)

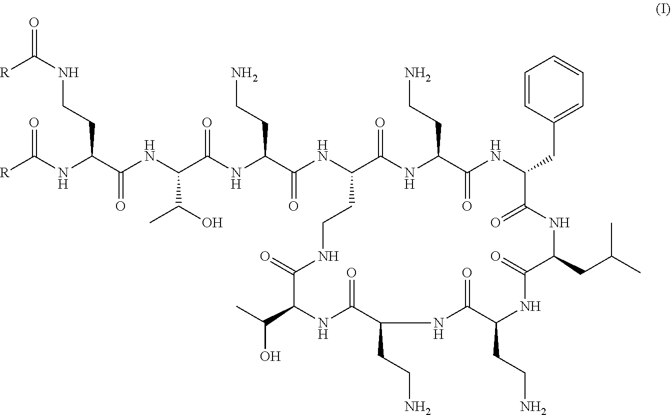

wherein: each R is independently a substituted or unsubstituted aliphatic lipid of C1-8, as an anti-microbial adjuvant, and an effective amount of the anti-microbial agent.

In another aspect of the invention, there is provided a method of determining if an adjuvant compound increases activity of an anti-microbial agent against a target micro-organism comprising co-administering to the target micro-organism the adjuvant compound and the anti-microbial agent, said adjuvant compound comprising a chemical structure as set forth in Formula (I)

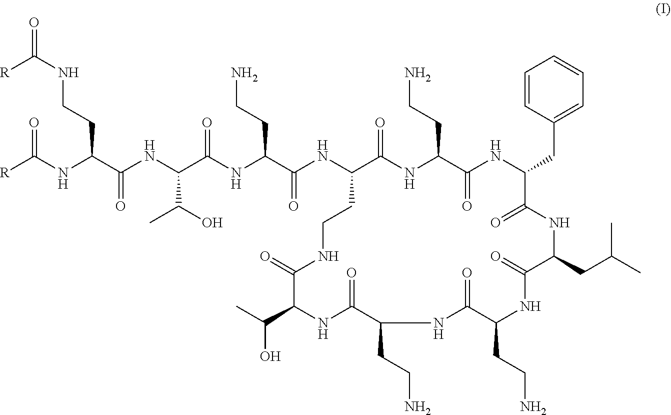

wherein: each R is independently a substituted or unsubstituted aliphatic lipid of C1-8;

determining activity of the anti-microbial agent and the adjuvant compound against the target micro-organism; and

comparing the activity to activity of an equivalent amount of the anti-microbial agent alone against the target micro-organism.

The invention will now be further explained and/or elucidated by way of examples; however, the invention is not necessarily limited to the examples.

### Example 1—Design and Synthesis of Dilipid Polymyxins

To study the effect of hydrophobicity on polymyxin's bioactivity, we prepared five dilipid polymyxins with varying lipid component (FIG. 2). Aside from an acylated N-terminus typically observed in polymyxins, we decided to incorporate another lipid to the amine side-chain of Dab1. Previous alanine scanning of the polymyxin structure suggested that the side-chain of Dab1 is not crucial for its activity [9]. We selected to install aliphatic hydrocarbon lipids with length of four- (1), eight- (2) or twelve- (3) carbons-long. We also included lipids that contain a bulky caged-hydrocarbon adamantyl group (4) and an aromatic bi-phenyl group (5) to explore the effect of hydrophobicity from non-aliphatic hydrocarbon chains.

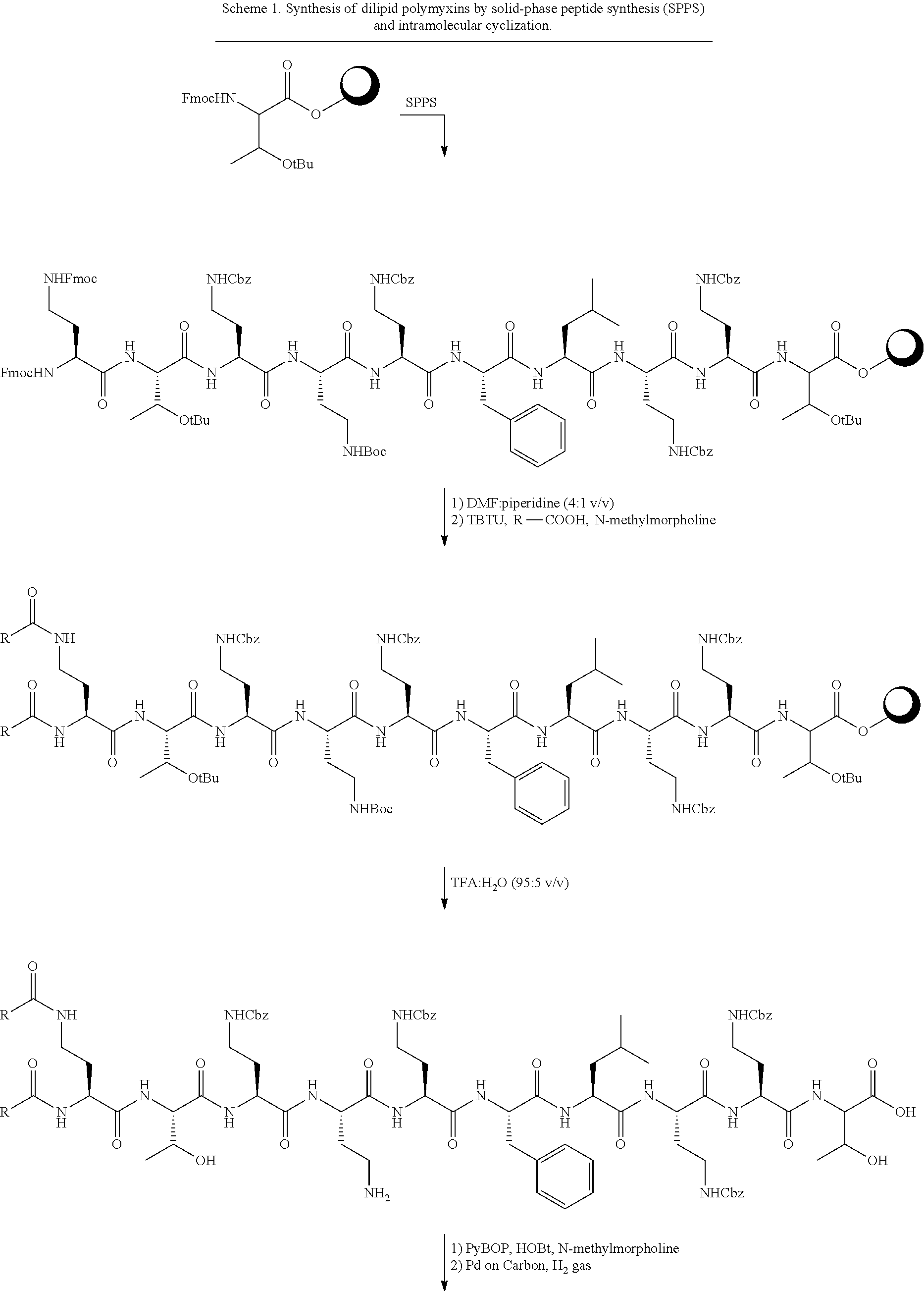

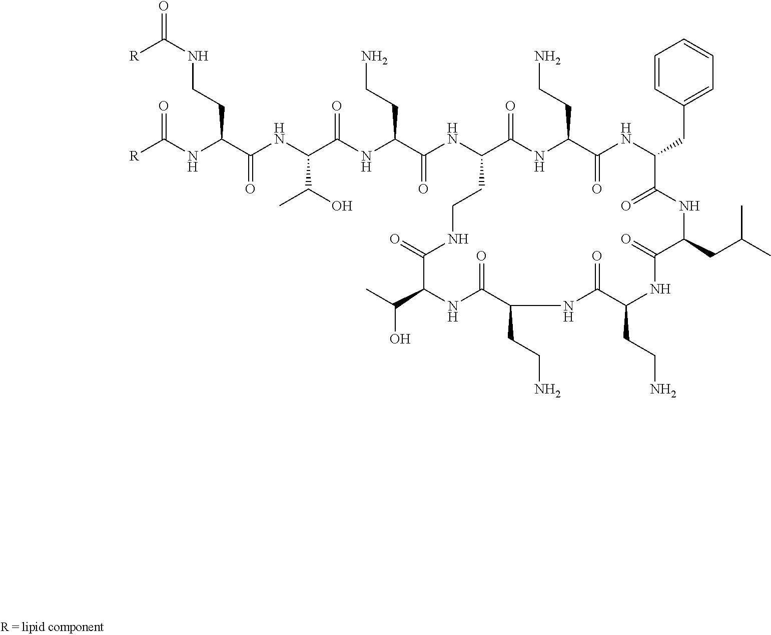

Solid-phase peptide synthesis followed by solution-phase intramolecular amide bond formation were performed to synthesize the dilipid polymyxins (Scheme 1) following our previously reported protocol with minor deviations [20]. Briefly, amino acids were immobilized on a Wang resin following a fluorenylmethyloxycarbonyl (Fmoc)-protection strategy. Dab containing an Fmoc-group at the Na and amine side-chain was immobilized in amino acid position 1. Fmoc deprotection of this Dab1 resulted to two free amine groups, which were then acylated with the lipid component. The resin was then subjected to an acidic solution of trifluoroacetic acid (TFA): water (95:5 v/v) to release the linear dilipid peptide with a free carboxyl C-terminus and amine side-chain of Dab at position 4 (Dab4). These two key functional groups were then reacted together to form an intramolecular amide bond that effectively cyclized the dilipid peptide. Removal of residual side-chain protecting groups yielded five dilipid polymyxins (FIG. 2).

### Example 2—Addition of Another Lipid Bestow Polymyxin Activity Against Gram-Positive Bacteria

The antibacterial activity of the dilipid polymyxins were evaluated against a panel of Gram-positive (Table 1) and Gram-negative bacteria (Table 2), some of which are MDR clinical isolates collected through the Canadian National Intensive Care Unit (CAN-ICU) surveillance study [21] or the Canadian Ward Surveillance (CANWARD) study [22]. Antibacterial activity was measured via minimum inhibitory concentration (MIC), which is the lowest concentration of the agent to inhibit bacterial growth. It was evident that the presence of a second lipid component bestow dilipid polymyxins superior activity against Gram-positive bacteria relative to colistin (Table 1). For instance, compounds 2 and 5 exhibited 32-fold better MIC than colistin against methicillin-resistant Staphylococcus aureus and methicillin-susceptible Staphylococcus epidermidis. This indicates that imparting another hydrophobic region within the polymyxin structure makes favorable interactions with lipoteichoic acid in Gram-positive bacterial membranes possible, presumably through hydrophobic effect, resulting in enhanced antibacterial activity.

### Example 3—. Dilipid Polymyxins Display Selective Antibacterial Activity Against P. aeruginosa Among Other Gram-Negative Bacteria

Evaluation of the dilipid polymyxins against Gram-negative bacteria revealed an interesting trend (Table 2). Specifically, addition of another lipid component to the amine side-chain of Dab1 reduced polymyxin's activity against Escherichia coli, P. aeruginosa, Stenotrophomonas maltophilia, A. baumannii, Klebsiella pneumoniae and Enterobacter cloacae. However, the reduction of activity against P. aeruginosa was not as drastic relative to other Gram-negative pathogens. For instance, polymyxin 2 diacylated with octanoic acid displayed only 4-8 fold lower MIC than colistin against all eleven P. aeruginosa strains tested (Table 2). An apparent hydrophobic threshold to retain antibacterial activity against P. aeruginosa was observed, where an eight carbons-long lipid as seen in 2 appeared to be optimal compared to shorter (four carbons-long in 1) or longer (twelve carbons-long in 3) lipid components. Dilipid polymyxins 4 and 5 that consist of caged adamantyl and aromatic biphenyl lipids, respectively, displayed 2-4 fold lower MIC against P. aeruginosa relative to 2. We believe that this selectivity for P. aeruginosa is due to differences in exopolysaccharides and lipopolysaccharides between Gram-negative bacteria. This finding may imply the possibility of developing antipseudomonal polymyxins for pathogen-specific therapy.

### Example 4—Addition of Another Lipid do not Enhance the Activity of Polymyxin Against Colistin-Resistant Gram-Negative Bacteria

We then addressed the possibility for the dilipid polymyxins to display enhanced antibacterial activity against colistin-resistant Gram-negative pathogens. Resistance to polymyxins are mainly due to outer membrane/LPS structural modifications (such as conjugation of ethanolamine and 4-aminoarabinose or lipid A deacylation) that result to reduced binding affinity, and consequently lowered antibacterial activity [23]. The extended hydrophobic region of the dilipid polymyxin may provide additional hydrophobic interactions to the outer membrane of colistin-resistant organisms. Eight MDR colistin-resistant Gram-negative bacterial isolates were tested, including two E. coli isolates harboring the mcr-1 plasmid-encoded polymyxin resistance gene [24]. We found no difference between the antibacterial activity of dilipid polymyxins and colistin against colistin-resistant Gram-negative bacteria (Table 3). Therefore, addition of a second lipid component to the polymyxin structure does not impart activity against colistin-resistant organisms.

### Example 5—Dilipid Polymyxins can Enhance the Activity of Other Antibiotics

The potential of the dilipid polymyxins to serve as an adjuvant in combination with other antibiotics was evaluated against P. aeruginosa PAO1 strain (Tables 4 and 7-12). We hypothesize that the ability to permeabilize the outer membrane, as seen in PMBN, is retained in dilipid polymyxins. Checkerboard assays were performed with the dilipid polymyxins in combination with twenty-one clinically-used antibiotics. The panel included representatives from the β-lactam, carbapenem, tetracycline, aminoglycoside and fluoroquinolone antibiotic families. Other agents that are used to treat Gram-negative bacterial infection such as fosfomycin, trimethoprim and chloramphenicol were also included. Moreover, agents with potent activity against Gram-positive bacteria but poor activity against Gram-negative bacteria such as novobiocin, vancomycin, clindamycin, linezolid, pleuromutilin and rifampicin were included. Fractional inhibitory concentration (FIC) index was calculated for each combination to measure potential synergism between the two agents. FIC was obtained by dividing the MIC of antibiotic/adjuvant in combination by the MIC of antibiotic/adjuvant alone, while FIC index was calculated by the summation of FIC indices for the antibiotic and adjuvant. An FIC index of ≤0.5, 0.5<x≤4, or >4 denotes for synergistic, additive or antagonistic interaction, respectively [25]. Initially, we assessed the adjuvant properties of PMBN against the panel of 21 antibiotics. PMBN potentiated all antibiotics tested except for those belonging to the aminoglycoside family (Table 4). Interestingly, dilipid polymyxin 1 that is diacylated with butyric acid exhibited similar adjuvant potency as PMBN (Table 4). Both 1 and PMBN possessed poor activity against P. aeruginosa alone (MIC of 128 μg/mL for both) but are able to enhance the activity of other antibiotics in combination presumably through outer membrane permeabilization. Since 1 and PMBN are structurally similar, this observation may imply that amino acid Dab1 and short lipid component of four carbons-long do not affect the ability of polymyxin to permeabilize the outer membrane. This finding may imply the development of polymyxin-based adjuvants where the Dab1 and lipid component are used as molecular scaffolds in appending further functional groups to modulate desired activity. However, not all the dilipid polymyxins potentiated the tested panel of antibiotics. For instance, the dilipid polymyxin 3 diacylated with twelve carbons-long and its isosteric bulky adamantyl counterpart 4 did not enhance the activity of any antibiotics. This may suggest that longer and bulky lipids may not be beneficial to the adjuvant properties of polymyxins.

### Example 6—Length of Lipid Component Affect the Ability of Polymyxins to Resist Efflux

To our curiosity, we explored whether efflux can affect the activity of polymyxins. It is widely accepted that polymyxins disrupt Gram-negative bacterial membranes that result to intracellular component leakage and bacterial cell death [4]. However, for this to occur requires the agent to reach the periplasmic space to interact and disrupt the inner membrane. Active efflux may potentially expel polymyxins out of the periplasm, effectively reducing its periplasmic and intracellular concentrations. To study the effect of efflux on polymyxins in Gram-negative pathogens, we used P. aeruginosa as they inherently overexpress efflux systems such as MexAB-OprM, MexCD-OprJ and MexXY-OprM. Moreover, antibiotic substrates for these efflux systems are mostly characterized [26,27]. We evaluated and compared the activity of the dilipid polymyxins, colistin and PMBN against wild-type PAO1 and two efflux-deficient P. aeruginosa isogenic mutants (Table 5). The strain PAO200 lacks the MexAB-OprM efflux system while strain PAO750 lacks five clinically-relevant pumps (MexAB-OprM, MexCD-OprJ, MexEF-OprN, MexJK and MexXY) and outer membrane protein OpmH. In agreement with a previous report [26], colistin was not affected by efflux as there was no difference in its MIC among the three P. aeruginosa strains tested (Table 4). Surprisingly, both 1 and PMBN appeared to be greatly affected by the MexAB-OprM efflux system (Table 4) as there was a 32- and 64-fold difference, respectively, in MIC of the agents against wild-type PAO1 and efflux-deficient PAO200. Deletion of other efflux systems seemed to not further alter the susceptibility of P. aeruginosa to 1 and PMBN as their MIC against PAO200 and PAO750 were comparable (Table 4). While not as potent compared to colistin (MIC of 0.5 μg/mL against PAO200), dilipid polymyxin 1 (MIC of 4 μg/mL against PAO200) and PMBN (MIC of 2 μg/mL against PAO200) displayed good antibacterial activity against MexAB-OprM deletion mutants. This suggests that 1 and PMBN may possess the ability to disrupt the inner membrane and/or have another periplasmic target but fall short in their ability to resist efflux. Our data implies that the length of the lipid component affected the ability of polymyxin to resist active efflux as derivatives of more than eight carbons-long (such as in colistin and 2) displayed no difference in MIC against the three P. aeruginosa strains tested. To our knowledge, this is the first reported evidence to show that active efflux in P. aeruginosa may affect the activity of a polymyxin derivative.

### Example 7—Length of Lipid Component Affect the Hemolytic Activity of Polymyxins

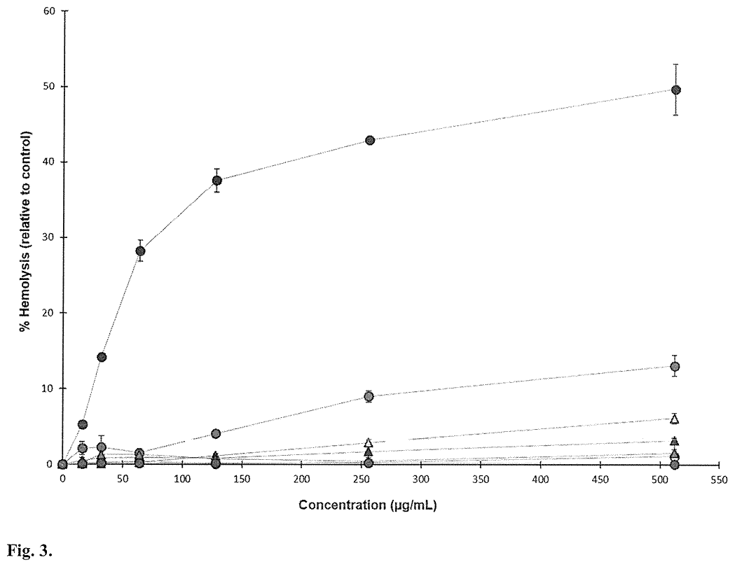

To address concerns of non-specific lysis that may arise from an enhanced hydrophobicity especially against eukaryotic cells, we evaluated the propensity of dilipid polymyxins to lyse eukaryotic red blood cells and compared them with colistin and PMBN. The length of hydrocarbon dilipid component was found to positively correlate with red blood cell hemolysis (FIG. 3). We quantified each compound's hemolytic activity by their MHC₅, or the minimum concentration to elicit 5% red blood cell hemolysis (Table 6). Compound 3 that is diacylated with twelve carbons-long lipid induced the most hemolysis (MHC₅ of 16 μg/mL) while compound 1 induced the least (MHC₅ of >512 μg/mL). In fact, the hemolytic activity of 1 resembles colistin and PMBN which show no hemolytic activity up to the highest concentration tested of 512 μg/mL (Table 6). While substantially non-hemolytic in comparison to 3, dilipid polymyxin 2 only elicted 6.2% hemolysis at 512 μg/mL. Interestingly, we found that the hemolysis induced by a twelve carbons-long aliphatic dilipid can be reduced by replacing the alkyl chain by isosteric caged or aromatic moieties. For instance, the bulky adamantyl dilipid seen in 4 (MHC₅ of >512 μg/mL) and the aromatic biphenyl dilipid seen in 5 (MHC₅ of 128 μg/mL) yielded significantly lower hemolysis compared to 3. Accordingly, the addition of either short-length aliphatic (eight carbons-long or less), caged or aromatic lipid is preferred for developing polymyxin-based agents that are non-hemolytic.

### Experimental

### Synthesis

### General Information

All reagents and solvents were purchased from common commercial suppliers and were used without further purifications otherwise stated. Synthesized compounds were purified, as specified in their synthesis, by reverse-phase flash chromatography using C18 silica gel (40-63 μm) purchased from Silicycle (USA). TLC was performed on silica gel 60 F254 (0.25 mm) obtained from Merck (USA) to check the presence of compound in each fractions and was visualized by both ultraviolet light and ninhydrin staining solution. The purity of all compounds were determined to be ≥95% via high-performance liquid chromatography (HPLC) analysis via Breeze HPLC Waters with 2998 PDA detector (1.2 nm resolution) coupled to Phenomenex Synergi Polar (50×2.0 mm) 4 μm reverse-phase column with phenyl ether-linked stationary phase. All purified compounds were characterized via 1-dimensional and 2-dimensional NMR experiments, to which NMR experiments were performed on a Bruker AMX-500 (500 MHz) instrument (Germany). The molecular weight for all synthesized compounds were recorded by Matrix-assisted Laser Desorption Ionization coupled to a time of flight mass analyzer and mass spectrometer (MALDI-TOF-MS) on a Bruker Ultraflextreme (Germany), using 2,5-dehydroxybenzoic acid as the matrix.

### Peptide Synthesis

Solid-phase peptide synthesis was performed following our previously reported protocol [20,28]. All linear diacylated peptides were synthesized on solid-support following an Fmoc-protection strategy. Wang p-alkoxybenzyl alcohol resin containing an already immobilized L-threonine was used to grow the peptide. Fmoc deprotection was done using a weak basic solution of N,N-dimethylformamide (DMF):piperidine (4:1 v/v). Peptide coupling reactions were performed by reacting the free amine of the immobilized amino acid with the free carboxylic acid of the incoming amino acid, via the peptide coupling reagent O-(benzotriazol-1-yl)-N,N,N′,N′-tetramethyluronium tetrafluoroborate (TBTU) (3 mol. equiv.) and N-methylmorpholine (3 mol. equiv.). Peptide coupling reactions were done in DMF with constant gentle agitation for 45 minutes. An acidic solution of TFA:water (95:5 v/v) was added to the resin and reacted for 30 minutes to cleave the peptide, followed by immediate evaporation in vacuo to afford the crude. MALDI-TOF-MS confirmed the presence and relative purity of the linear diacylated peptides.

### Intramolecular Cyclization and Deprotection

The linear diacylated peptide was then mixed with benzotriazole-1-yl-oxy-trispyrrolidino-phosphonium hexafluorophosphate (PyBOP) (4 mol. equiv.), hydroxybenzotriazole (HOBt) (4 mol. equiv.), and N-methymorpholine (10 mol. equiv.) in anhydrous DMF under very dilute conditions, to which was then vigorously stirred for 2 h to induce intramolecular cyclization via amide bond formation between the carboxyl end of L-threonine at position 10 (Thr10) and the amine side-chain of Dab4. The solvent was then removed in vacuo. The cyclized diacylated peptide was precipitated from the crude by addition of cold water. It was then filtered to obtain a pale brown solid. Dichloromethane (DCM) was added to result to a partially dissolve product and co-distilled in vacuo to dryness. MALDI-TOF-MS was then used to confirm the product. The cyclized diacylated peptide was then subjected to catalytic hydrogenolysis. The compound was dissolved in a mixture of 4:5:1 methanol/acetic acid/water. Palladium on carbon was then added, followed by H₂ gas (balloon) to remove the remaining carboxybenzyl (Cbz) protecting groups. The resulting solution was then filtered via Nylon filter, to which the filter was then washed with methanol. The filtrate was evaporated to dryness in vacuo. The crude was purified via reverse-phase flash chromatography with an eluent mixture of water and methanol (both solvents spiked with 0.1% TFA), following a gradient of 0% to 50% methanol in water ratio (2.5% stepwise), to afford compounds 1-5.

### Dibutyric Acid (Di-C4) Polymyxin (1)

¹H NMR (500 MHz, Methanol-d₄) δ: 7.32-7.19 (m, 5H D-Phe₆ aromatic), 4.51 (dd, J=8.7, 4.4 Hz 1H, Dab_{5α}), 4.49-4.24 (m, 7H, Dab_{1α}+Dab_{3α}+Dab_{4α}+Dab_{8α}+Dab_{8α}+D-Phe_{6α}+Thr_{2β}), 4.21 (d, J=3.2 Hz, 1H, Thr_{2α}), 4.20-4.16 (m, 1H, Thr_{10β}), 4.11 (dd, J=11.5, 3.8 Hz, 1H, Leu_{7α}), 4.06 (d, J=4.9 Hz, 1H, Thr_{10α}), 3.67-3.57 (m, 1H, Dab_{4γ1}), 3.42-3.34 (m, 1H, Dab_{4γ2}), 3.23-2.85 (m, 12H, Dab_{1γ2}+Dab_{3γ}+Dab_{8γ}+Dab_{9γ}+Dab_{4γ2}+D-Phe_{6β}+Dab_{5γ}), 2.52-2.42 (m, 1H, Dab_{3β1}), 2.31-1.90 (m, 14H, Dab_{1β1}+Dab_{3β2}+Dab_{8β}+Dab_{9β}+Dab_{4β}+Dab_{5β}+Di-C₄ CO-CH₂—CH₂—CH₃), 1.84-1.75 (m, 1H, Dab_{1β2}), 1.69-1.59 (m, 4H, Di-C₄ CO—CH₂-CH₂—CH₃), 1.54-1.46 (m, 1H Leu_{7β1}), 1.40-1.31 (m, 1H Leu_{7β2}), 1.29-1.15 (m, 6H, Thr_{2γ}+Thr_{10γ}), 0.98-0.91 (m, 6H, Di-C₄ CH₃), 0.88-0.58 (m, 7H, Leu_{7γ}+Leu_{7δ}). ¹³C NMR (126 MHz, Methanol-d₄) δ: 175.60, 175.09, 173.87, 173.40, 172.60, 172.41, 172.33, 172.14, 171.83, 171.46, 171.32, 170.85, 135.84 (D-Phe₆ C without H), 128.85, 128.34, 126.72, 66.28, 65.77, 60.21, 59.32, 56.42, 51.94, 51.90, 51.84, 51.66, 51.49, 50.70, 49.92, 39.29, 37.64 (Di-C₄ CO—CH₂—CH₂—CH₃), 37.21 (Di-C₄ CO—CH₂—CH₂—CH₃), 36.64, 36.61, 36.44, 36.36, 35.96, 35.58, 34.90, 30.81, 30.25, 30.02, 28.70, 28.39, 23.52, 22.77, 22.17, 20.01, 19.15, 18.99 (Di-C₄ CO—CH₂—CH₂—CH₃), 18.88 (Di-C₄ CO—CH₂—CH₂—CH₃), 18.79, 12.67 (Di-C₄ CO—CH₂—CH₂—CH₃), 12.63 (Di-C₄ CO—CH₂—CH₂—CH₃). MALDI-TOF-MS m/z calcd for C₅₅H₉₅N₁₆O₁₄ (M+H)⁺ monoisotopic peak: 1203.721; found 1203.717.

### Dioctanoic Acid (Di-C₈) Polymyxin (2)

¹H NMR (500 MHz, Methanol-d₄) δ 7.31-7.19 (m, 5H, D-Phe₆ aromatic), 4.51 (dd, J=8.8, 4.4 Hz, 1H, Dab_{5α}), 4.46-4.38 (m, 2H, Dab_{1α}+Dab_{3α}), 4.38-4.24 (m, 5H, Dab_{4α}+Dab_{8α}+Dab_{8α}+D-Phe_{δa}+Thr_{2β}), 4.24-4.20 (m, 1H, Thr_{2α}), 4.20-4.14 (m, 1H, Thr_{10β}), 4.14-4.09 (m, 1H, Leu_{7α}), 4.06 (d, J=3.1 Hz, 1H, Thr_{10α}), 3.67-3.53 (m, 1H, Dab_{4γ1}), 3.42-3.34 (m, 1H, Dab_{1γ1}i, 3.23-3.12 (m, 2H, Dab_{1γ2}+Dab_{4γ2}), 3.12-2.87 (m, 10H, Dab_{3γ}+Dab_{8γ}+Dab_{9γ}+D-Phe_{6β}+Dab_{5γ}), 2.53-2.41 (m, 1H, Dab_{3β1}), 2.33-2.16 (m, 7H, Dab_{1β1}+Dab_{5β}+Di-C₈ CO-CH₂—), 2.16-1.92 (m, 7H, Dab_{3β2}+Dab_{8β}+Dab_{9β}+Dab_{4β}), 1.82-1.73 (m, 1H, Dab_{1β2}), 1.67-1.56 (m, 4H, Di-C₈ CO—CH₂-CH₂—), 1.54-1.46 (m, 1H, Leu_{7β1}), 1.40-1.25 (m, 17H, Leu_{7β2}+Di-C₈ alkyl), 1.25-1.08 (m, 6H, Thr_{2γ}+Thr_{10γ}), 0.89 (t, J=6.6 Hz, 6H, Di-C₈ CH₃), 0.84-0.78 (m, 1H, Leu_{7γ}), 0.78-0.57 (m, 6H, Leu_{7δ}). ¹³C NMR (126 MHz, Methanol-d₄) δ 175.68, 175.29, 173.88, 173.36, 172.59, 172.40, 172.38, 172.12, 171.82, 171.45, 171.32, 170.82, 135.85 (D-Phe₆ C without H), 128.85, 128.34, 126.72, 66.32, 65.79, 59.30, 56.39, 51.81, 51.67, 51.58, 51.48, 51.45, 49.91, 48.19, 48.02, 47.85, 47.68, 39.33, 39.30, 36.64, 36.61, 36.47, 36.43, 36.36, 35.96 (Di-C₈ CO—CH₂—), 35.80 (Di-C₈ CO—CH₂—), 35.62, 35.38, 34.92, 31.46, 30.85, 30.24, 30.01, 29.99, 28.93, 28.86, 28.71, 28.37, 25.68 (Di-C₈ CO—CH₂—CH₂—), 25.65 (Di-C₈ CO—CH₂—CH₂—), 25.57, 23.56, 23.53, 22.25, 22.16, 20.02, 19.16, 18.78, 13.01 (Di-C₈ CH₃), 12.97 (Di-C₈ CH₃). MALDI-TOF-MS m/z calcd for C₆₃H₁₁₁N₁₆O₁₄ (M+H)⁺ monoisotopic peak: 1315.846; found 1315.865.

### Didodecanoic Acid (Di-C₁₂) Polymyxin (3)

¹H NMR (500 MHz, Methanol-d₄) δ: 7.31-7.17 (m, 5H, D-Phe₆ aromatic), 4.59-4.50 (m, 1H, Dab_{5α}), 4.50-4.26 (m, 7H, Dab_{1α}+Dab_{3α}+Dab_{4α}+Dab_{8α}+D-Phe_{6α}+Thr_{2β}+Dab_{9α}), 4.23 (d, J=3.4 Hz, 1H, Thr_{2α}), 4.21-4.15 (m, 1H, Thr_{10β}), 4.15-4.09 (m, 1H, Leu_{7α}), 4.03 (d, J=3.7 Hz, 1H, Thr_{10α}), 3.73-3.58 (m, 1H, Dab_{4γ1}), 3.41-3.34 (m, 1H, Dab_{1γ1}), 3.21-2.82 (m, 12H, Dab_{1γ2}+Dab_{4γ2}+Dab_{3γ}+Dab_{8γ}+Dab_{9γ}+D-Phe_{6β}+Dab_{5γ}), 2.76-2.67 (m, 1H), 2.51-2.36 (m, 1H, Dab_{3β1}), 2.33-1.93 (m, 14H. Dab_{1β1}+Dab_{5β}+Di-C₁₂ CO-CH₂—+Dab_{3β2}+Dab_{8β}+Dab_{9β}+Dab_{4β}), 1.85-1.78 (m, 1H, Dab_{1β2}), 1.64-1.56 (m, 4H, Di-C₁₂ CO—CH₂-CH₂—), 1.54-1.47 (m, 1H, Leu_{7β1}), 1.46-1.41 (m, 1H, Leu_{7β2}), 1.34-1.25 (m, 32H, Di-C₁₂ alkyl), 1.23-1.13 (m, 6H, Thr_{2γ}+Thr_{10γ}), 0.95-0.63 (m, 13H, Di-C₁₂ CH₃+, Leu_{7γ}+Leu_{7δ}). ¹³C NMR (126 MHz Methanol-d₄) δ: 175.68, 175.30, 173.89, 173.37, 173.10, 172.80, 172.58, 172.18, 171.84, 171.47, 171.32, 171.24, 129.05 (D-Phe₆ C without H), 128.90, 128.88, 128.34, 128.23, 126.72, 76.43, 75.43, 74.83, 73.01, 66.35, 65.79, 59.30, 56.43, 51.60, 51.46, 49.98, 49.64, 39.30, 36.67, 36.48, 36.45 (Di-C₁₂ CO—CH₂—), 36.36 (Di-C₁₂ CO—CH₂—), 35.81, 35.64, 35.40, 31.63, 29.33, 29.31, 29.23, 29.06, 29.03, 28.98, 28.66, 25.69 (Di-C₁₂ CO—CH₂—CH₂—), 25.66 (Di-C₁₂ CO—CH₂—CH₂—), 25.57, 23.53, 22.29, 22.17, 20.02, 19.40, 19.16, 18.79, 13.00 (Di-C₁₂ CH₃), 12.99 (Di-C₁₂ CH₃) MALDI-TOF-MS m/z calcd for C₇₁H₁₂₇N₁₆O₁₄ (M+H)⁺ monoisotopic peak: 1427.971; found 1428.006

### Diadamantane Acetic Acid (Di-Adamantyl) Polymyxin (4)

¹H NMR (500 MHz, Methanol-d₄) δ: 7.32-7.20 (m, 5H, D-Phe₆ aromatic), 4.51 (dd, J=8.7, 4.3 Hz, 1H, Dab_{5α}), 4.47-4.32 (m, 5H, Dab_{1α}+Dab_{3α}+Dab_{4α}+Dab_{8α}+D-Phe_{6α}), 4.32-4.26 (m, 2H, Dab_{9α}+Thr_{2β}), 4.25-4.22 (m, Thr_{2α} 1H), 4.22-4.15 (m, 1H, Thr_{10β}), 4.12 (dd, J=11.5, 3.4 Hz, 1H, Leu_{7α}), 4.07 (d, J=4.6 Hz, 1H, Thr_{10α}), 3.69-3.54 (m, 1H, Dab_{4γ1}), 3.43-3.35 (m, 1H, Dab_{1γ1}), 3.19-2.86 (m, 12H, Dab_{1γ2}+Dab_{4γ2}+Dab_{3γ}+Dab_{5γ}+Dab_{8γ}+Dab_{9γ}+D-Phe_{6β}), 2.55-2.41 (m, 1H, Dab_{3β1}), 2.30-2.20 (m, 2H, Dab_{8β}), 2.20-2.05 (m, 5H, Dab_{1β1}+Dab_{5β1}+Dab_{3β2}+Dab_{9β}), 2.04-1.91 (m, 13H, Dab_{5β2}+Dab_{4β}+Di-adamantyl CO-CH₂—+Di-adamantyl —CH—), 1.82-1.76 (m, 1H, Dab_{1β2}), 1.75-1.59 (m, 24H, Di-adamantyl —CH₂—), 1.54-1.47 (m, 1H, Leu_{7β1}), 1.39-1.32 (m, 1H, Leu_{7β2}), 1.29-1.15 (m, 6H, Thr_{2γ}+Thr_{10γ}), 1.00-0.86 (m, 1H, Leu_{7γ}), 0.82-0.62 (m, 6H, Leu_{7δ}). ¹³C NMR (126 MHz, Methanol-d₄) δ: 173.89, 173.31, 173.20, 172.85, 172.62, 172.47, 172.33, 172.26, 171.91, 171.56, 171.39, 171.30, 135.82 (D-Phe₆ C without H), 128.88, 128.35, 128.18, 126.72, 126.67, 66.43, 65.71, 59.36, 59.12, 56.45, 51.96, 51.87, 51.64, 51.54, 50.59 (Di-adamantyl CO—CH₂), 50.55 (Di-adamantyl CO—CH₂), 49.99, 42.40 (Di-adamantyl —CH₂—), 42.30 (Di-adamantyl —CH₂—), 39.26, 36.67, 36.43, 36.40, 36.01, 35.68, 34.93, 32.67 (Di-adamantyl —C—), 32.48 (Di-adamantyl —C—), 31.02, 28.73 (Di-adamantyl —CH—), 28.71 (Di-adamantyl —CH—), 23.49, 22.20, 20.03, 19.16, 18.89. MALDI-TOF-MS m/z calcd for C₇₁H₁₁₅N₁₆O₁₄ (M+H)⁺ monoisotopic peak: 1415.877; found 1415.865

### Dibiphenyl-4-Carboxylic Acid (Di-Biphenyl) Polymyxin (5)

¹H NMR (500 MHz, Methanol-d₄) δ: 8.01-7.89 (m, 4H, Di-biphenyl aromatic), 7.76-7.57 (m, 8H, Di-biphenyl aromatic), 7.47-7.36 (m, 6H, Di-biphenyl aromatic), 7.29-7.18 (m, 5H, D-Phe₆ aromatic), 4.73-4.66 (m, 1H, Dab_{5α}), 4.52-4.39 (m, 3H, Dab_{1α}+Dab_{3α}+D-Phe_{6α}), 4.40-4.24 (m, 5H, Dab_{4α}+Dab_{8α}+Dab_{9α}+Thr_{2β}+Thr_{2α}), 4.21-4.10 (m, 2H, Thr_{10β}+Leu_{7α}), 4.05 (d, J=4.8 Hz, 1H, Thr_{10α}), 3.78-3.71 (m, 1H, Dab_{1γ1}), 3.69-3.60 (m, 1H, Dab_{4γ1}), 3.57-3.51 (m, 1H, Dab_{1γ2}), 3.19-2.88 (m, 11H, Dab_{4γ2}+Dab_{3γ}+Dab_{5γ}+Dab_{8γ}+Dab_{9γ}+D-Phe_{6β}), 2.50-2.41 (m, 1H, Dab_{3β1}), 2.39-2.31 (m, 2H, Dab_{8β}), 2.30-2.25 (m, 1H, +Dab_{5β1}), 2.22-2.11 (m, 4H, Dab_{1β1}+Dab_{3β2}+Dab_{4β}), 2.04-1.92 (m, 3H, Dab_{5β2}+Dab_{9β}), 1.87-1.80 (m, 1H, Dab_{1β2}), 1.54-1.47 (m, 1H, Leu_{7β1}), 1.38-1.32 (m, 1H, Leu_{7β2}), 1.26-1.11 (m, 6H, Thr_{2γ}+Thr_{10γ}), 0.95-0.87 (m, 1H, Leu_{7γ}), 0.82-0.60 (m, 6H, Leu_{7δ}). ¹³C NMR (126 MHz, Methanol-d₄) δ: 173.91, 173.81, 173.71, 173.68, 173.51, 172.44, 171.48, 171.46, 171.27, 170.94, 169.31 (Di-biphenyl carbonyl), 168.81 (Di-biphenyl carbonyl), 144.81 (Di-biphenyl C without H), 144.77 (Di-biphenyl C without H), 144.39 (Di-biphenyl C without H), 144.35 (Di-biphenyl C without H), 139.71 (Di-biphenyl C without H), 139.64 (Di-biphenyl C without H), 135.82 (D-Phe₆ C without H), 132.66, 132.62, 131.88, 128.90, 128.86, 128.63, 128.62, 128.34, 128.20, 127.92, 127.81, 127.72, 127.58, 126.68, 119.99, 117.70, 117.65, 115.36, 115.30, 66.37, 66.35, 59.67, 59.58, 56.22, 52.66, 51.91, 51.89, 51.87, 51.53, 51.11, 50.96, 45.93, 39.29, 36.73, 36.65, 36.61, 36.56, 36.44, 36.39, 36.00, 30.66, 30.13, 29.30, 28.94, 28.69, 28.43, 25.88, 23.54, 23.31, 22.17, 20.02, 19.13, 18.80. MALDI-TOF-MS m/z calcd for C₇₃H₉₉N₁₆O₁₄ (M+H)⁺ monoisotopic peak: 1423.752; found 1423.767

### Biological Studies

Bacterial strains were either obtained from the American Type Culture Collection (ATCC), Canadian National Intensive Care Unit (CAN-ICU) surveillance study [21] or the Canadian Ward Surveillance (CANWARD) study [22]. Strains from both CAN-ICU and CANWARD are isolates recovered from patients diagnosed with a presumed infectious disease that were admitted in a participating medical center across Canada. Efflux-deficient strain PAO200 (lacking MexAB-OprM efflux system) and PA0750 (lacking MexAB-OprM, MexCD-OprJ, MexEF-OprN, MexJK and MexXY efflux systems, and the outer membrane protein OpmH) were kindly gifted by Dr. Ayush Kumar (University of Manitoba).

### Antimicrobial Susceptibility Assay

The in vitro antibacterial activity of the compounds studied were assessed by broth microdilution susceptibility testing following CLSI guidelines [29]. Bacterial cultures were grown overnight prior to the assay. The overnight grown cultures were diluted in saline to 0.5 McFarland turbidity, followed by 1:50 dilution in Mueller-Hinton broth (MHB) for inoculation to a final concentration of approximately 5×10⁵ colony forming units/mL. Testing was performed on 96-well plates where the tested compounds were 2-fold serially diluted in MHB and incubated with equal volumes of bacterial inoculum at 37° C. for 18 h. The MIC was determined as the lowest concentration of the compound to inhibit visible bacterial growth in the form of turbidity, to which was confirmed via an EMax Plus microplate reader (Molecular Devices, USA) at a wavelength of 590 nm. Wells containing MHB with or without bacterial cells were used as positive or negative control, respectively.

### Checkerboard Assay

The assay was performed on 96-well plates as previously described [20,30]. Briefly, the antibiotic at study was 2-fold serially diluted along the x-axis, while the adjuvant was 2-fold serially diluted along the y-axis to create a matrix in which each well consists of a combination of both at different concentrations. Overnight grown bacterial cultures were diluted in saline to 0.5 McFarland turbidity, followed by 1:50 dilution in MHB and inoculation on each well to a final concentration of approximately 5×10⁵ colony forming units/mL. Wells containing only MHB with or without bacterial cells were used as positive or negative control, respectively. Plates were incubated at 37° C. for 18 h and examined for visible turbidity, which was confirmed via an EMax Plus microplate reader (Molecular Devices, USA) at a wavelength of 590 nm. The fractional inhibitory concentration (FIC) of the antibiotic was calculated via dividing the MIC of antibiotic in the presence of adjuvant by the MIC of antibiotic alone. Similarly, the FIC of adjuvant was calculated via dividing the MIC of adjuvant in the presence of antibiotic by the MIC of adjuvant alone. The FIC index was obtained by the summation of both FIC values. The FIC index was then interpreted as synergistic, additive, or antagonistic for values of ≤0.5, 0.5<x<4, or ≥4, respectively [25].

### Hemolytic Assay

The ability of the compounds to lyse eukaryotic red blood cells was measured by the amount of hemoglobin released upon incubation with pig erythrocytes, following a published protocol [30]. Briefly, fresh pig blood (generously provided by Dr. Richard Hodges, Director of Animal Care and Veterinary Services of the University of Manitoba) drawn from a pig's antecubital vein was centrifuged at 1000 g for 5 min at 4° C., washed with phosphate-buffered saline (PBS) three times, and re-suspended in the same buffer, consecutively. Compounds at study were 2-fold serially diluted in PBS on a 96-well plate and mixed with equal volumes of erythrocyte solution. Post 1 h incubation at 37° C., the intact cells were pelleted by centrifugation at 1000 g for 5 min at 4° C. Resulting supernatant was then transferred to a new 96-well plate. The hemoglobin released was then measured via an EMax Plus microplate reader (Molecular Devices, USA) at 570 nm wavelength. Erythrocytes in PBS with or without 0.1% Triton X-100 were used as positive or negative control, respectively.

The scope of the claims should not be limited by the preferred embodiments set forth in the examples but should be given the broadest interpretation consistent with the description as a whole.

### REFERENCES

[1] J. M. Pogue, J. K. Ortwine, K. S. Kaye, Clinical considerations for optimal use of the polymyxins: A focus on agent selection and dosing, Clin. Microbiol. Infect. 23 (2017) 229-233. doi:10.1016/j.cmi.2017.02.023.
[2] J. M. Pogue, J. K. Ortwine, K. S. Kaye, Are there any ways around the exposure-limiting nephrotoxicity of the polymyxins?, Int. J. Antimicrob. Agents. 48 (2016) 622-626. doi:10.1016/j.ijantimicag.2016.11.001.
[3] O. Zusman, S. Altunin, F. Koppel, Y. Dishon Benattar, H. Gedik, M. Paul, Polymyxin monotherapy or in combination against carbapenem-resistant bacteria: systematic review and meta-analysis, J. Antimicrob. Chemother. 72 (2017) 29-39. doi:10.1093/jac/dkw377.
[4] T. Velkov, P. E. Thompson, R. L. Nation, J. Li, Structure-activity relationships of polymyxin antibiotics, J. Med. Chem. 53 (2010) 1898-1916. doi:10.1021/jm900999h.
[5] T. Velkov, R. L. Soon, P. L. Chong, J. X. Huang, M. A. Cooper, M. A. K. Azad, M. A. Baker, P. E. Thompson, K. Roberts, R. L. Nation, A. Clements, R. A. Strugnell, J. Li, Molecular basis for the increased polymyxin susceptibility of Klebsiella pneumoniae strains with under-acylated lipid A, Innate Immun. 19 (2013) 265-277. doi:10.1177/1753425912459092.
[6] R. A. Dixon, I. Chopra, Polymyxin B and polymyxin B nonapeptide alter cytoplasmic membrane permeability in Escherichia coli, J. Antimicrob. Chemother. 18 (1986) 557-563.
[7] Z. Z. Denis, J. D. Swarbrick, K. D. Roberts, M. A. K. Azad, J. Akter, A. S. Home, R. L. Nation, K. L. Rogers, P. E. Thompson, T. Velkov, J. Li, Probing the penetration of antimicrobial polymyxin lipopeptides into Gram-negative bacteria, Bioconjug. Chem. 25 (2014) 750-760. doi:10.1021/bc500094d.
[8] M. G. Scott, M. R. Gold, R. E. Hancock, Interaction of cationic peptides with lipoteichoic acid and gram-positive bacteria, Infect. Immun. 67 (1999) 6445-6453.
[9] K. Kanazawa, Y. Sato, K. Ohki, K. Okimura, Y. Uchida, M. Shindo, N. Sakura, Contribution of each amino acid residue in polymyxin B(3) to antimicrobial and lipopolysaccharide binding activity, Chem. Pharm. Bull. (Tokyo). 57 (2009) 240-244. doi:10.1248/cpb.57.240.
[10] A. Gallardo-Godoy, C. Muldoon, B. Becker, A. G. Elliott, L. H. Lash, J. X. Huang, M. S. Butler, R. Pelingon, A. M. Kavanagh, S. Ramu, W. Phetsang, M. A. T. Blaskovich, M. A. Cooper, Activity and predicted nephrotoxicity of synthetic antibiotics based on polymyxin B, J. Med. Chem. 59 (2016) 1068-1077. doi:10.1021/acs.jmedchem.5b01593.
[11] R. L. Danner, K. A. Joiner, M. Rubin, W. H. Patterson, N. Johnson, K. M. Ayers, J. E. Parrillo, Purification, toxicity, and antiendotoxin activity of polymyxin B nonapeptide, Antimicrob. Agents Chemother. 33 (1989) 1428-1434.
[12] I. Ofek, S. Cohen, R. Rahmani, K. Kabha, D. Tamarkin, Y. Herzig, E. Rubinstein, Antibacterial synergism of polymyxin B nonapeptide and hydrophobic antibiotics in experimental gram-negative infections in mice, Antimicrob. Agents Chemother. 38 (1994) 374-377.
[13] H. Tsubery, I. Ofek, S. Cohen, M. Fridkin, Structure-function studies of polymyxin B nonapeptide: implications to sensitization of gram-negative bacteria, J. Med. Chem. 43 (2000) 3085-3092.
[14] H. Tsubery, I. Ofek, S. Cohen, M. Eisenstein, M. Fridkin, Modulation of the hydrophobic domain of polymyxin B nonapeptide: effect on outer-membrane permeabilization and lipopolysaccharide neutralization, Mol. Pharmacol. 62 (2002) 1036-1042.
[15] M. Vaara, O. Siikanen, J. Apajalahti, J. Fox, N. Frimodt-Moller, H. He, A. Poudyal, J. Li, R. L. Nation, T. Vaara, A novel polymyxin derivative that lacks the fatty acid tail and carries only three positive charges has strong synergism with agents excluded by the intact outer membrane, Antimicrob. Agents Chemother. 54 (2010) 3341-3346. doi:10.1128/AAC.01439-09.
[16] R. Domalaon, T. Idowu, G. G. Zhanel, F. Schweizer, Antibiotic hybrids: the next generation of agents and adjuvants against Gram-negative pathogens?, Clin. Microbiol. Rev. 31 (2018). doi:10.1128/CMR.00077-17.
[17] D. Corbett, A. Wise, T. Langley, K. Skinner, E. Trimby, S. Birchall, A. Dorali, S. Sandiford, J. Williams, P. Warn, M. Vaara, T. Lister, Potentiation of antibiotic activity by a novel cationic peptide: potency and spectrum of activity of SPR741, Antimicrob. Agents Chemother. 61 (2017). doi:10.1128/AAC.00200-17.
[18] M.-L. Han, T. Velkov, Y. Zhu, K. D. Roberts, A. P. Le Brun, S. H. Chow, A. D. Gutu, S. M. Moskowitz, H.-H. Shen, J. Li, Polymyxin-induced lipid A deacylation in Pseudomonas aeruginosa perturbs polymyxin penetration and confers high-level resistance, ACS Chem. Biol. 13 (2018) 121-130. doi:10.1021/acschembio.7b00836.
[19] Y. Kinoshita, F. Yakushiji, H. Matsui, H. Hanaki, S. Ichikawa, Study of the structure-activity relationship of polymyxin analogues, Bioorg. Med. Chem. Lett. (2018). doi:10.1016/j.bmc1.2018.03.028.
[20] R. Domalaon, X. Yang, Y. Lyu, G. G. Zhanel, F. Schweizer, Polymyxin B3-tobramycin hybrids with Pseudomonas aeruginosa-selective antibacterial activity and strong potentiation of rifampicin, minocycline, and vancomycin, ACS Infect. Dis. 3 (2017) 941-954. doi:10.1021/acsinfecdis.7b00145.
[21] G. G. Zhanel, M. DeCorby, N. Laing, B. Weshnoweski, R. Vashisht, F. Tailor, K. a. Nichol, A. Wierzbowski, P. J. Baudry, J. a. Karlowsky, P. Lagace-Wiens, A. Walkty, M. McCracken, M. R. Mulvey, J. Johnson, D. J. Hoban, Antimicrobial-resistant pathogens in intensive care units in Canada: results of the Canadian National Intensive Care Unit (CAN-ICU) study, 2005-2006, Antimicrob. Agents Chemother. 52 (2008) 1430-1437. doi:10.1128/AAC.01538-07.
[22] G. G. Zhanel, H. J. Adam, M. R. Baxter, J. Fuller, K. a. Nichol, A. J. Denisuik, P. R. S. Lagacé-Wiens, A. Walkty, J. a. Karlowsky, F. Schweizer, D. J. Hoban, Canadian Antimicrobial Resistance Alliance, Antimicrobial susceptibility of 22746 pathogens from Canadian hospitals: results of the CANWARD 2007-11 study, J. Antimicrob. Chemother. 68 Suppl 1 (2013) 7-22. doi:10.1093/jac/dkt022.
[23] T. Velkov, Z. Z. Denis, J. X. Huang, M. A. K. Azad, M. Butler, S. Sivanesan, L. M. Kaminskas, Y.-D. Dong, B. Boyd, M. A. Baker, M. A. Cooper, R. L. Nation, J. Li, Surface changes and polymyxin interactions with a resistant strain of Klebsiella pneumoniae, Innate Immun. 20 (2014) 350-363. doi:10.1177/1753425913493337.
[24] A. Walkty, J. A. Karlowsky, H. J. Adam, P. Lagace-Wiens, M. Baxter, M. R. Mulvey, M. McCracken, S. M. Poutanen, D. Roscoe, G. G. Zhanel, Frequency of MCR-1-mediated colistin resistance among Escherichia coli clinical isolates obtained from patients in Canadian hospitals (CANWARD 2008-2015), C. Open. 4 (2016) E641-E645. doi:10.9778/cmajo.20160080.
[25] J. Meletiadis, S. Pournaras, E. Roilides, T. J. Walsh, Defining fractional inhibitory concentration index cutoffs for additive interactions based on self-drug additive combinations, Monte Carlo simulation analysis, and in vitro-in vivo correlation data for antifungal drug combinations against Aspergillus fumi, Antimicrob. Agents Chemother. 54 (2010) 602-609. doi:10.1128/AAC.00999-09.
[26] N. Masuda, E. Sakagawa, S. Ohya, N. Gotoh, H. Tsujimoto, T. Nishino, Substrate specificities of MexAB-OprM, MexCD-OprJ, and MexXY-oprM efflux pumps in Pseudomonas aeruginosa, Antimicrob. Agents Chemother. 44 (2000) 3322-3327.
[27] R. Chuanchuen, C. T. Narasaki, H. P. Schweizer, The MexJK efflux pump of Pseudomonas aeruginosa requires OprM for antibiotic efflux but not for efflux of triclosan, J. Bacteriol. 184 (2002) 5036-5044.
[28] R. Domalaon, X. Yang, J. O'Neil, G. G. Zhanel, N. Mookherjee, F. Schweizer, Structure-activity relationships in ultrashort cationic lipopeptides: the effects of amino acid ring constraint on antibacterial activity, Amino Acids. 46 (2014) 2517-2530. doi:10.1007/s00726-014-1806-z.
[29] The Clinical and Laboratory Standards Institute, Performance Standards for Antimicrobial Susceptibility Testing CLSI supplement M100S 26th ed., Clin. Lab. Stand. Institute, Wayne, Pa. (2016).
[30] R. Domalaon, Y. Sanchak, L. C. Koskei, Y. Lyu, G. G. Zhanel, G. Arthur, F. Schweizer, Short proline-rich lipopeptide potentiates minocycline and rifampin against multidrug- and extensively drug-resistant Pseudomonas aeruginosa, Antimicrob. Agents Chemother. 62 (2018). doi:10.1128/AAC.02374-17.

TABLE 1
Antimicrobial activity of dilipid polymyxins against
laboratory reference and multidrug-resistant
clinical isolates of Gram-positive bacteria

| | MIC, μg/mL | | | | | |
|---|---|---|---|---|---|---|
| Organism | 1 | 2 | 3 | 4 | 5 | Colistin |
| S. aureus ATCC 29213 | >128 | 16 | >128 | 16 | 8 | 256 |
| MRSA^{a} ATCC 33592 | >128 | 16 | >128 | 32 | 16 | 512 |
| MSSE^{b} CANWARD-2008 81388 | >128 | 8 | 64 | 16 | 8 | 256 |
| MRSE^{c} CAN-ICU 61589 | >128 | 16 | 64 | 16 | 8 | 128 |
| E. faecalis ATCC 29212 | >128 | 32 | 64 | 32 | 16 | 256 |
| E. faecium ATCC 27270 | >128 | 16 | 8 | 16 | 8 | 256 |
| S. pneumoniae ATCC 49619 | >128 | 32 | 64 | 32 | 32 | 512 |

^{a}methicillin-resistant Staphylococcus aureus
^{b}methicillin-susceptible Staphylococcus epidermidis
^{c}methicillin-resistant S. epidermidis.

TABLE 2
Antimicrobial activity of dilipid polymyxins against
laboratory reference and multidrug-resistant
clinical isolates of Gram-negative bacteria

| | MIC, μg/mL | | | | | |
|---|---|---|---|---|---|---|
| Organism | 1 | 2 | 3 | 4 | 5 | Colistin |
| E. coli ATCC 25922 | >128 | 4 | 16 | 4 | 8 | 0.5 |
| E. coli CAN-ICU 61714 | >128 | 32 | 128 | 64 | 32 | 0.5 |
| E. coli CAN-ICU 63074 | >128 | 64 | 64 | 64 | 32 | ≤0.5 |
| E. coli | >128 | 64 | 64 | 64 | 32 | NT |
| CANWARD-2011 97615 | | | | | | |
| E. coli 107115 | 128 | 2 | 8 | 4 | 4 | 0.5 |
| P. aeruginosa ATCC 27853 | >128 | 8 | >128 | 16 | 32 | 2 |
| P. aeruginosa PAO1 | 128 | 4 | 32 | 4 | 8 | 1 |
| P. aeruginosa | >128 | 8 | 64 | 16 | 16 | 2 |
| CAN-ICU 62308 | | | | | | |
| P. aeruginosa | >128 | 8 | >128 | 32 | 64 | NT |
| CANWARD-2011 96846 | | | | | | |
| P. aeruginosa | 64 | 2 | 16 | 8 | 8 | 0.25 |
| PA259-96918 | | | | | | |
| P. aeruginosa | 2 | 2 | 32 | 4 | 8 | 0.5 |
| PA260-97103 | | | | | | |
| P. aeruginosa | >128 | 8 | 32 | 8 | 8 | 2 |
| PA262-101856 | | | | | | |
| P. aeruginosa | 128 | 4 | 64 | 8 | 8 | 2 |
| PA264-104354 | | | | | | |
| P. aeruginosa PA095 | 64 | 2 | 8 | 4 | 4 | 0.25 |
| P. aeruginosa | >128 | 4 | 32 | 8 | 8 | 1 |
| PA100036 | | | | | | |
| P. aeruginosa PA101885 | 128 | 4 | 32 | 8 | 8 | 1 |
| S. maltophilia | >128 | 32 | 128 | 64 | 64 | 64 |
| CAN-ICU 62584 | | | | | | |
| A. baumannii ATCC 17978 | >128 | 32 | >128 | 32 | 16 | 0.25 |
| A. baumannii | >128 | 64 | 128 | 128 | 64 | 32 |
| CAN-ICU 63169 | | | | | | |
| A. baumannii 110193 | >128 | 16 | >128 | 32 | 16 | 0.25 |
| A. baumannii LAC-4 | 128 | 16 | >128 | 16 | 16 | 0.125 |
| A. baumannii AB027 | >128 | 32 | 128 | 64 | 6 | 0.25 |
| A. baumannii AB031 | >128 | 8 | 64 | 16 | 8 | 0.25 |
| K. pneumoniae | >128 | >128 | >128 | >128 | >128 | ≤0.25 |
| ATCC 13883 | | | | | | |
| K. pneumoniae 116381 | >128 | >128 | >128 | >128 | >128 | 1 |
| E. cloacae 117029 | 64 | 2 | 16 | 4 | 8 | 0.125 |

TABLE 3
Antimicrobial activity of dilipid polymyxins against colistin-
resistant multidrug-resistant Gram-negative bacterial isolates

| | MIC, μg/mL | | | | | |
|---|---|---|---|---|---|---|
| Organism | 1 | 2 | 3 | 4 | 5 | Colistin |
| E. coli 94393 | >64 | 4 | 32 | 16 | 16 | 4 |
| E. coli 94474 | >64 | 4 | >64 | 8 | 8 | 16 |
| P. aeruginosa 91433 | >128 | 4 | 8 | 8 | 8 | 4 |
| P. aeruginosa 101243 | >128 | 32 | 16 | 64 | >128 | 1024 |
| P. aeruginosa 114228 | >128 | 4 | >128 | 8 | 16 | 4 |
| A. baumannii 92247 | >64 | 8 | 32 | 8 | 8 | 4 |
| K. pneumoniae 113250 | >64 | >64 | >64 | >64 | >64 | 256 |
| K. pneumoniae 113254 | >64 | >64 | >64 | >64 | >64 | 256 |

TABLE 4
Evaluation for synergism of dilipid polymyxins or
polymyxin B nonapeptide (PMBN) and clinically-used antibiotics
against P. aeruginosa PAO1^{a}

| | FIC index | | | | | |
|---|---|---|---|---|---|---|
| Drug | 1 | 2 | 3 | 4 | 5 | PMBN |
| Ceftazidime | 0.37 | 1.00 | 1.00 | 0.53 | 0.62 | 0.19 |
| Piperacillin | 0.75 | 0.51 | 1.00 | 1.03 | 1.00 | 0.31 |
| Aztreonam | 0.37 | 0.53 | 0.75 | 0.51 | 0.62 | 0.16 |
| Meropenem | 0.50 | 0.56 | 1.00 | 1.00 | 1.00 | 0.37 |
| Doripenem | 0.75 | 0.62 | 1.00 | 0.56 | 1.00 | 0.31 |
| Minocycline | 0.08 | 0.50 | 0.62 | 0.75 | 0.62 | 0.09 |
| Doxycycline | 0.08 | 0.51 | 0.62 | 1.00 | 0.53 | 0.05 |
| Tobramycin | 1.00 | 1.03 | 1.01 | 1.01 | 1.01 | 1.00 |
| Streptomycin | 0.75 | 0.75 | 1.01 | 1.03 | 1.00 | 0.50^{c} |
| Gentamicin | 1.00 | 2.01 | 1.01 | 1.03 | 2.01 | 1.50 |
| Moxifloxacin | 0.25 | 1.50 | 1.00 | 1.50 | 0.75 | 0.37 |
| Ciprofloxacin | 0.50 | 0.75 | 1.01 | 1.03 | 1.00 | 0.50 |
| Fosfomycin | 0.25 | 0.51 | 0.75 | 0.75 | 0.56 | 0.37 |
| Trimethoprim | 0.25 | 0.56 | 0.62 | 1.00 | 0.62 | 0.09 |
| Chloramphenicol | 0.09 | 0.37 | 0.62 | 0.62 | 0.50 | 0.07 |
| Novobiocin | 0.09 | 0.26 | 0.50^{b} | 0.50 | 0.50 | 0.04 |
| Vancomycin | 0.28 | 0.51 | 1.00 | 0.75 | 0.51 | 0.02 |
| Clindamycin | 0.09 | 0.31 | 0.56 | 0.62 | 0.53 | 0.03 |
| Linezolid | 0.09 | 0.50^{b} | 1.00 | 1.00 | 0.53 | 0.06 |
| Pleuromutilin | 0.07 | 0.51 | 0.51 | 0.51 | 0.50^{c} | 0.03 |
| Rifampicin | 0.02 | 0.75 | 0.75 | 0.51 | 0.31 | 0.01 |

^{a}yellow shaded box indicate FIC index of ≤0.5 therefore is synergistic
^{b}FIC index is 0.500977 therefore not synergistic
^{c}FIC index is 0.503906 therefore not synergistic

TABLE 5
Antimicrobial activity of dilipid polymyxins, colistin and polymyxin
B nonapeptide (PMBN) against efflux-deficient P. aeruginosa

| | MIC, μg/mL | | | | | | |
|---|---|---|---|---|---|---|---|
| Organism | 1 | 2 | 3 | 4 | 5 | Colistin | PMBN |
| P. aeruginosa PAO1^{a} | 128 | 4 | 32 | 4 | 8 | 1 | 128 |
| P. aeruginosa PAO200^{b} | 4 | 2 | 32 | 4 | 4 | 0.5 | 2 |
| P. aeruginosa PAO750^{c} | 8 | 2 | 32 | 8 | 4 | 0.5 | 4 |

^{a}wild type
^{b}efflux-deficient strain that lacks the MexAB-OprM pump
^{c}efflux-deficient strain that lacks five clinically-relevant pumps (MexAB-OprM, MexCD-OprJ, MexEF-OprN, MexJK and MexXY) and outer membrane protein OpmH

TABLE 6
Hemolytic properties of dilipid polymyxins,
colistin and polymyxin B nonapeptide (PMBN)

| | | % Hemolysis at 512 |
|---|---|---|
| Polymyxin | MHC₅^{a} | μg/mL of polymyxin |
| 1 | >512 | 1.1 ± 1.6 |
| 2 | 512 | 6.2 ± 0.7 |
| 3 | 16 | 49.7 ± 3.3 |
| 4 | >512 | 3.2 ± 0.4 |
| 5 | 128 | 13.1 ± 1.4 |
| colistin | >512 | 1.6 ± 0.5 |
| PMBN | >512 | 1.1 ± 0.1 |

^{a}minimum concentration (μg/mL) that resulted in 5% red blood cell hemolysis

TABLE 7
Evaluation for synergism of 1 and clinically-
used antibiotics against P. aeruginosa PAO1

| | MIC_{Drug} | | MIC₁ | | | |
|---|---|---|---|---|---|---|
| | (MIC_{Drug} | | (MIC₁ | | FIC | Interpre- |
| Drug | in combo) | | in combo) | | index | tation |
| Ceftazidime | 2 | (0.25) | 128 | (32) | 0.37 | Synergy |
| Piperacillin | 4 | (2) | 128 | (32) | 0.75 | Additive |
| Aztreonam | 4 | (0.5) | 128 | (32) | 0.37 | Synergy |
| Meropenem | 1 | (0.25) | 128 | (32) | 0.50 | Synergy |
| Doripenem | 1 | (0.5) | 128 | (32) | 0.75 | Additive |
| Minocycline | 16 | (1) | 128 | (2) | 0.08 | Synergy |
| Doxycycline | 8 | (0.5) | 128 | (2) | 0.08 | Synergy |
| Tobramycin | 1 | (1) | 128 | (0.5) | 1.00 | Additive |
| Streptomycin | 16 | (8) | 128 | (32) | 0.75 | Additive |
| Gentamicin | 2 | (2) | 128 | (0.0625) | 1.00 | Additive |
| Moxifloxacin | 1 | (0.125) | 128 | (16) | 0.25 | Synergy |
| Ciprofloxacin | 0.25 | (0.0625) | 128 | (32) | 0.50 | Synergy |
| Fosfomycin | 16 | (2) | 128 | (16) | 0.25 | Synergy |
| Trimethoprim | 128 | (16) | 128 | (16) | 0.25 | Synergy |
| Chloram- | 32 | (2) | 128 | (4) | 0.09 | Synergy |
| phenicol | | | | | | |
| Novobiocin | 512 | (16) | 128 | (8) | 0.09 | Synergy |
| Vancomycin | 256 | (8) | 128 | (32) | 0.28 | Synergy |
| Clindamycin | 1024 | (32) | 128 | (8) | 0.09 | Synergy |
| Linezolid | 1024 | (64) | 128 | (4) | 0.09 | Synergy |
| Pleuromutilin | 512 | (4) | 128 | (8) | 0.07 | Synergy |
| Rifampicin | 16 | (0.125) | 128 | (2) | 0.02 | Synergy |

TABLE 8
Evaluation for synergism of 2 and clinically-
used antibiotics against P. aeruginosa PAO1

| | MIC_{Drug} | | MIC₂ | | | |
|---|---|---|---|---|---|---|
| | (MIC_{Drug} | | (MIC₂ | | FIC | Interpre- |
| Drug | in combo) | | in combo) | | index | tation |
| Ceftazidime | 2 | (1) | 4 | (2) | 1.00 | Additive |
| Piperacillin | 4 | (0.0625) | 8 | (4) | 0.51 | Additive |
| Aztreonam | 4 | (0.125) | 8 | (4) | 0.53 | Additive |
| Meropenem | 1 | (0.0625) | 8 | (4) | 0.56 | Additive |
| Doripenem | 1 | (0.125) | 8 | (4) | 0.62 | Additive |
| Minocycline | 16 | (4) | 8 | (2) | 0.50 | Synergy |
| Doxycycline | 8 | (0.125) | 8 | (4) | 0.51 | Additive |
| Tobramycin | 1 | (1) | 4 | (0.125) | 1.03 | Additive |
| Streptomycin | 16 | (4) | 8 | (4) | 0.75 | Additive |
| Gentamicin | 2 | (4) | 8 | (0.125) | 2.01 | Additive |
| Moxifloxacin | 1 | (1) | 4 | (2) | 1.50 | Additive |
| Ciprofloxacin | 0.25 | (0.0625) | 8 | (4) | 0.75 | Additive |
| Fosfomycin | 16 | (0.125) | 8 | (4) | 0.51 | Additive |
| Trimethoprim | 128 | (8) | 8 | (4) | 0.56 | Additive |
| Chloram- | 32 | (4) | 8 | (2) | 0.37 | Synergy |
| phenicol | | | | | | |
| Novobiocin | 512 | (8) | 8 | (2) | 0.26 | Synergy |
| Vancomycin | 256 | (2) | 8 | (4) | 0.51 | Additive |
| Clindamycin | 1024 | (64) | 8 | (2) | 0.31 | Synergy |
| Linezolid | 1024 | (1) | 8 | (4) | 0.50^{a} | Additive |
| Pleuromutilin | 512 | (4) | 4 | (2) | 0.51 | Additive |
| Rifampicin | 16 | (8) | 4 | (1) | 0.75 | Additive |

^{a}FIC index is 0.500977 therefore not synergistic

TABLE 9
Evaluation for synergism of 3 and clinically-
used antibiotics against P. aeruginosa PAO1

| | MIC_{Drug} | | MIC₃ | | | |
|---|---|---|---|---|---|---|
| | (MIC_{Drug} | | (MIC₃ | | FIC | Interpre- |
| Drug | in combo) | | in combo) | | index | tation |
| Ceftazidime | 2 | (1) | 32 | (16) | 1.00 | Additive |
| Piperacillin | 4 | (2) | 32 | (16) | 1.00 | Additive |
| Aztreonam | 4 | (1) | 32 | (16) | 0.75 | Additive |
| Meropenem | 1 | (0.5) | 32 | (16) | 1.00 | Additive |
| Doripenem | 1 | (0.5) | 32 | (16) | 1.00 | Additive |
| Minocycline | 16 | (2) | 32 | (16) | 0.62 | Additive |
| Doxycycline | 8 | (1) | 32 | (16) | 0.62 | Additive |
| Tobramycin | 1 | (1) | 32 | (0.25) | 1.01 | Additive |
| Streptomycin | 16 | (16) | 32 | (0.25) | 1.01 | Additive |
| Gentamicin | 2 | (2) | 32 | (0.25) | 1.01 | Additive |
| Moxifloxacin | 1 | (0.5) | 32 | (16) | 1.00 | Additive |
| Ciprofloxacin | 0.125 | (0.125) | 32 | (0.25) | 1.01 | Additive |
| Fosfomycin | 16 | (8) | 32 | (8) | 0.75 | Additive |
| Trimethoprim | 128 | (16) | 32 | (16) | 0.62 | Additive |
| Chloram- | 32 | (4) | 32 | (16) | 0.62 | Additive |
| phenicol | | | | | | |
| Novobiocin | 512 | (0.5) | 32 | (16) | 0.50^{a} | Additive |
| Vancomycin | 256 | (128) | 32 | (16) | 1.00 | Additive |
| Clindamycin | 1024 | (64) | 32 | (16) | 0.56 | Additive |
| Linezolid | 1024 | (0.25) | 32 | (32) | 1.00 | Additive |
| Pleuromutilin | 512 | (8) | 32 | (16) | 0.51 | Additive |
| Rifampicin | 16 | (8) | 32 | (8) | 0.75 | Additive |

^{a}FIC index is 0.500977 therefore not synergistic

TABLE 10
Evaluation for synergism of 4 and clinically-
used antibiotics against P. aeruginosa PAO1

| | MIC_{Drug} | | MIC₄ | | | |
|---|---|---|---|---|---|---|
| | (MIC_{Drug} | | (MIC₄ | | FIC | Interpre- |
| Drug | in combo) | | in combo) | | index | tation |
| Ceftazidime | 2 | (0.0625) | 16 | (8) | 0.53 | Additive |
| Piperacillin | 4 | (4) | 8 | (0.25) | 1.03 | Additive |
| Aztreonam | 4 | (0.0625) | 16 | (8) | 0.51 | Additive |
| Meropenem | 1 | (0.5) | 8 | (4) | 1.00 | Additive |
| Doripenem | 1 | (0.0625) | 16 | (8) | 0.56 | Additive |
| Minocycline | 16 | (4) | 8 | (4) | 0.75 | Additive |
| Doxycycline | 8 | (4) | 8 | (4) | 1.00 | Additive |
| Tobramycin | 1 | (1) | 16 | (0.25) | 1.01 | Additive |
| Streptomycin | 16 | (16) | 8 | (0.25) | 1.03 | Additive |
| Gentamicin | 2 | (2) | 8 | (0.25) | 1.03 | Additive |
| Moxifloxacin | 1 | (1) | 8 | (4) | 1.50 | Additive |
| Ciprofloxacin | 0.125 | (0.125) | 8 | (0.25) | 1.03 | Additive |
| Fosfomycin | 32 | (8) | 8 | (4) | 0.75 | Additive |
| Trimethoprim | 128 | (0.25) | 8 | (8) | 1.00 | Additive |
| Chloram- | 32 | (4) | 8 | (4) | 0.62 | Additive |
| phenicol | | | | | | |
| Novobiocin | 512 | (2) | 8 | (4) | 0.50 | Additive |
| Vancomycin | 256 | (64) | 8 | (4) | 0.75 | Additive |
| Clindamycin | 1024 | (128) | 8 | (4) | 0.62 | Additive |
| Linezolid | 1024 | (0.25) | 8 | (8) | 1.00 | Additive |
| Pleuromutilin | 512 | (4) | 8 | (4) | 0.51 | Additive |
| Rifampicin | 16 | (0.25) | 8 | (4) | 0.51 | Additive |

TABLE 11
Evaluation for synergism of 5 and clinically-
used antibiotics against P. aeruginosa PAO1

| | MIC_{Drug} | | MIC₅ | | | |
|---|---|---|---|---|---|---|
| | (MIC_{Drug} | | (MIC₅ | | FIC | Interpre- |
| Drug | in combo) | | in combo) | | index | tation |
| Ceftazidime | 2 | (0.25) | 16 | (8) | 0.62 | Additive |
| Piperacillin | 4 | (2) | 16 | (8) | 1.00 | Additive |
| Aztreonam | 4 | (0.5) | 16 | (8) | 0.62 | Additive |
| Meropenem | 1 | (0.5) | 16 | (8) | 1.00 | Additive |
| Doripenem | 1 | (0.5) | 16 | (8) | 1.00 | Additive |
| Minocycline | 16 | (2) | 16 | (8) | 0.62 | Additive |
| Doxycycline | 8 | (0.25) | 16 | (8) | 0.53 | Additive |
| Tobramycin | 1 | (1) | 16 | (0.25) | 1.01 | Additive |
| Streptomycin | 16 | (8) | 16 | (8) | 1.00 | Additive |
| Gentamicin | 2 | (4) | 16 | (0.25) | 2.01 | Additive |
| Moxifloxacin | 1 | (0.25) | 16 | (8) | 0.75 | Additive |
| Ciprofloxacin | 0.25 | (0.125) | 16 | (8) | 1.00 | Additive |
| Fosfomycin | 16 | (1) | 16 | (8) | 0.56 | Additive |
| Trimethoprim | 128 | (16) | 16 | (8) | 0.62 | Additive |
| Chloram- | 32 | (8) | 16 | (4) | 0.50 | Synergy |
| phenicol | | | | | | |
| Novobiocin | 512 | (128) | 16 | (4) | 0.50 | Synergy |
| Vancomycin | 256 | (2) | 16 | (8) | 0.51 | Additive |
| Clindamycin | 1024 | (32) | 16 | (8) | 0.53 | Additive |
| Linezolid | 1024 | (32) | 16 | (8) | 0.53 | Additive |
| Pleuromutilin | 512 | (2) | 16 | (8) | 0.50^{a} | Additive |
| Rifampicin | 16 | (1) | 16 | (4) | 0.31 | Synergy |

^{a}FIC index is 0.503906 therefore not synergistic

TABLE 12
Evaluation for synergism of polymyxin B nonapeptide (PMBN) and
clinically-used antibiotics against P. aeruginosa PAO1

| | MIC_{Drug} | | MIC_{PMBN} | | | |
|---|---|---|---|---|---|---|
| | (MIC_{Drug} | | (MIC_{PMBN} | | FIC | Interpre- |
| Drug | in combo) | | in combo) | | index | tation |
| Ceftazidime | 2 | (0.25) | 128 | (8) | 0.19 | Synergy |
| Piperacillin | 4 | (1) | 128 | (8) | 0.31 | Synergy |
| Aztreonam | 4 | (0.5) | 128 | (4) | 0.16 | Synergy |
| Meropenem | 0.5 | (0.125) | 128 | (16) | 0.37 | Synergy |
| Doripenem | 1 | (0.25) | 128 | (8) | 0.31 | Synergy |
| Minocycline | 16 | (0.5) | 128 | (8) | 0.09 | Synergy |
| Doxycycline | 16 | (0.25) | 128 | (4) | 0.05 | Synergy |
| Tobramycin | 1 | (1) | 128 | (0.5) | 1.00 | Additive |
| Streptomycin | 16 | (8) | 128 | (0.5) | 0.50^{a} | Additive |
| Gentamicin | 2 | (2) | 128 | (64) | 1.50 | Additive |
| Moxifloxacin | 1 | (0.125) | 128 | (32) | 0.37 | Synergy |
| Ciprofloxacin | 0.125 | (0.0312) | 128 | (32) | 0.50 | Synergy |
| Fosfomycin | 16 | (2) | 128 | (32) | 0.37 | Synergy |
| Trimethoprim | 128 | (8) | 128 | (4) | 0.09 | Synergy |
| Chloram- | 32 | (2) | 128 | (1) | 0.07 | Synergy |
| phenicol | | | | | | |
| Novobiocin | 512 | (4) | 128 | (4) | 0.04 | Synergy |
| Vancomycin | 256 | (4) | 128 | (1) | 0.02 | Synergy |
| Clindamycin | 1024 | (32) | 128 | (0.5) | 0.03 | Synergy |
| Linezolid | 1024 | (32) | 128 | (4) | 0.06 | Synergy |
| Pleuromutilin | 512 | (16) | 128 | (0.5) | 0.03 | Synergy |
| Rifampicin | 16 | (0.0625) | 128 | (0.5) | 0.01 | Synergy |

^{a}FIC index is 0.503906 therefore not synergistic

1. A compound comprising a chemical structure as set forth in formula (I): 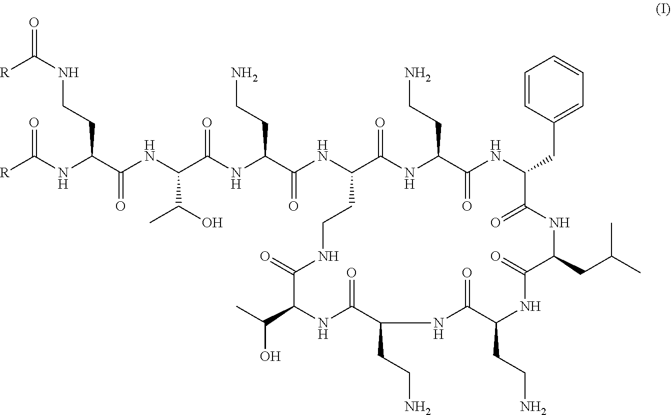
wherein: each R is independently a substituted or unsubstituted aliphatic lipid of C1-8.

2. The compound according to claim 1 wherein the substituted or unsubstituted aliphatic lipid is C1-C7.

3. The compound according to claim 1 wherein the aliphatic lipids are substituted by replacing the alkyl chains with isosteric caged or aromatic moieties.

4. The compound according to claim 1 where the compound is for use as an anti-bacterial adjuvant.

5. The compound according to claim 4 wherein the compound is used in an anti-infective composition.

6. (canceled)

7. (canceled)

8. (canceled)

9. (canceled)

10. A method of treating a microbial infection comprising: administering to an individual in need of such treatment an effective amount of an antimicrobial agent and an effective amount of a compound comprising a chemical structure as set forth in Formula (I): 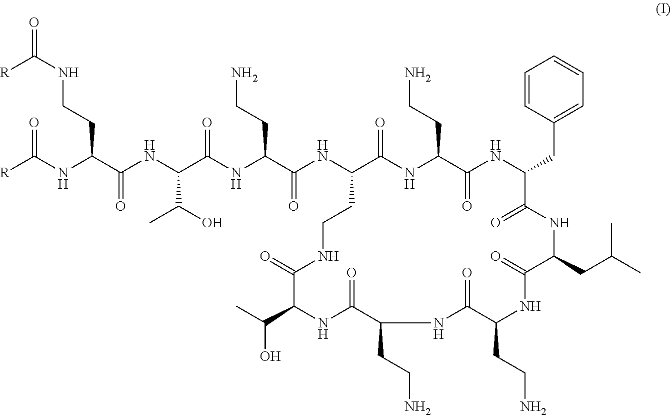
wherein: each R is independently a substituted or unsubstituted aliphatic lipid of C1-8.

11. The method according to claim 10 wherein the person in need of such treatment is an individual who is known to have or who is suspected of having a microbial infection.

12. The method according to claim 10 wherein the substituted or unsubstituted aliphatic lipid is C1-C7.

13. The method according to claim 10 wherein the aliphatic lipids are substituted by replacing the alkyl chains with isosteric caged or aromatic moieties.

14. A method of increasing activity of an anti-microbial agent against a target micro-organism comprising co-administering to the micro-organism a compound comprising a chemical structure as set forth in Formula (I) 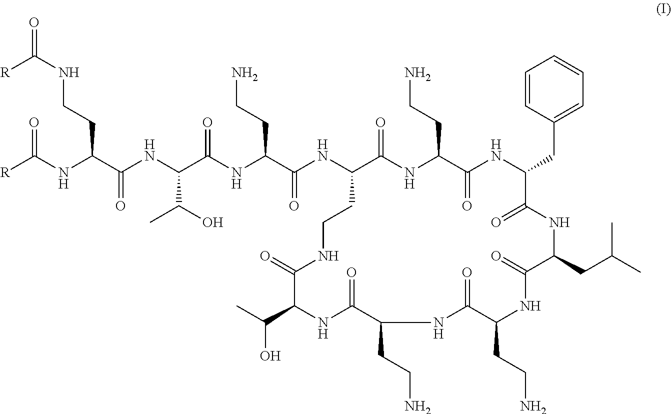
wherein: each R is independently a substituted or unsubstituted aliphatic lipid of C1-8, as an anti-microbial adjuvant, and an effective amount of the anti-microbial agent.

15. The method according to claim 14 wherein the substituted or unsubstituted aliphatic lipid is C1-C7.

16. The method according to claim 14 wherein the aliphatic lipids are substituted by replacing the alkyl chains with isosteric caged or aromatic moieties.
